(19) United States
(12) Patent Application Publication
(10) Pub. No.: US 2007/0037011 A1
(43) Pub. Date: Feb. 15, 2007

(54) Arylamine compound and synthetic method thereof

(75) Inventors: Harue Nakashima, Atsugi (JP); Sachiko Kawakami, Isehara (JP)

Correspondence Address: ERIC ROBINSON, PMB 955, 21010 SOUTHBANK ST., POTOMAC FALLS, VA, 20165, US

(73) Assignee: Semiconductor Energy Laboratory Co., Atsugi-shi (JP)

(21) Appl. No.: 11/500,278

(22) Filed: Aug. 8, 2006

(30) Foreign Application Priority Data
Aug. 12, 2005 (JP) 2005-234432

(51) Int. Cl. H01L 51/54; H05B 33/14; C07C 211/00; C07D 209/88

(52) U.S. Cl. 428/690; 428/917; 313/504; 257/E51.049; 257/E51.051; 548/440; 548/442; 564/429; 564/434

(57) ABSTRACT

It is an object of the present invention to provide an arylamine compound which has resistance to repeated oxidation reactions. The present invention provides a secondary arylamine compound represented by General Formula 1.
(In the formula, Ar¹¹ is an aryl group having 7 to 25 carbon atoms or a heteroaryl group having 7 to 25 carbon atoms. Ar¹² and Ar¹³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms.)

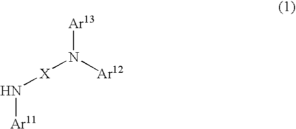

### TECHNICAL FIELD

The present invention relates to an arylamine compound and a synthetic method thereof. In addition, the present invention relates to a material for a light emitting element obtained by using an arylamine compound, and a light emitting element and an electronic device manufactured by using the material for a light emitting element.

### BACKGROUND ART

In recent years, research and development of a light emitting element using a light-emitting organic compound have been actively carried out. A basic structure of the light emitting element is such that a layer containing a light-emitting organic compound is sandwiched between a pair of electrodes. By applying a voltage to this element, electrons and holes are injected into the layer containing a light-emitting organic compound from the pair of electrodes so that a current flows. Then, the carriers (electrons and holes) are recombined, so that the light-emitting organic compound forms an excited state, and when the excited state returns to a ground state, light is emitted. With such a mechanism, such a light emitting element is called a current excitation type light emitting element.

In addition, there is a singlet excited state or a triplet excited state as an excitation state formed by the organic compound, and light emitted in the case of the singlet excited state is called fluorescence, and light emitted in the case of the triplet excited state is called phosphorescence.

A great advantage of such a light emitting element is that the light emitting element can be manufactured to be thin and lightweight because the light emitting element is made of an organic thin film, for example, having a thickness of approximately 0.1 μm. In addition, extremely high response speed is another advantage because the time between carrier injection and light emission is approximately 1 μsec or less. These characteristics are considered suitable for a flat panel display element.

Such a light emitting element is formed in a film shape. Thus, plane emission can be easily obtained by forming a large-area element. This characteristic is difficult to be obtained by using a point light source typified by an incandescent lamp or an LED or by using a line light source typified by a fluorescent lamp. Therefore, the above-described light emitting element also has a high utility value as a planar light source which is applicable to lighting or the like.

There are many problems related to materials in improving characteristics of the light emitting element. Therefore, improvement of an element structure, development of a material, and the like are conducted in order to overcome these problems.

As a cause of deterioration of a current excitation type light emitting element, there is deterioration of a material included in a layer containing a light emitting substance, which is provided between a pair of electrodes. In the current excitation type light emitting element, a material included in the layer containing a light emitting substance repeats an oxidation reaction and a reduction reaction by a current flowing in the layer containing a light emitting substance. When a material which is easily decomposed by the oxidation reaction or the reduction reaction is contained in the layer containing a light emitting substance, the material gradually deteriorates by the repeated oxidation reactions or repeated reduction reactions, and the light emitting element itself deteriorates.

### DISCLOSURE OF INVENTION

In view of the above-described problem, it is an object of the present invention to provide an arylamine compound which has resistance to repeated oxidation reactions.

It is another object of the present invention to provide a synthesizing method of an arylamine compound which has resistance to repeated oxidation reactions.

It is still another object of the present invention to provide a material for a light emitting element, which is obtained by using the arylamine compound having resistance to the repeated oxidation reactions, and a light emitting element and an electronic device which are manufactured by using the material for a light emitting element.

One feature of the present invention is a secondary arylamine compound represented by General Formula 1. 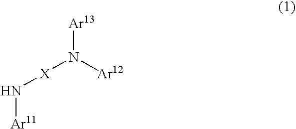
(In the formula, Ar¹¹ is an aryl group having 7 to 25 carbon atoms or a heteroaryl group having 7 to 25 carbon atoms. Ar¹² and Ar¹³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms.)

One feature of the present invention is a secondary arylamine compound represented by General Formula 2. 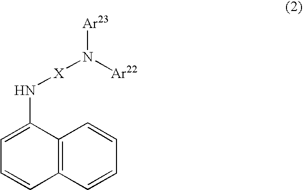
(In the formula, Ar²² and Ar²³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms.)

One feature of the present invention is a secondary arylamine compound represented by Structural Formula 31.

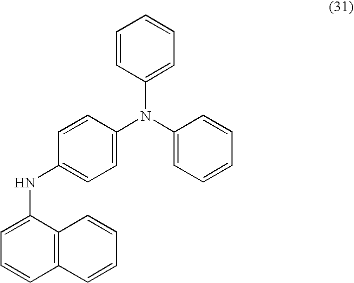

One feature of the present invention is a material for a light emitting element, which has the above-described secondary arylamine compound as a substituent.

One feature of the present invention is a material for a light emitting element, represented by General Formula 4. 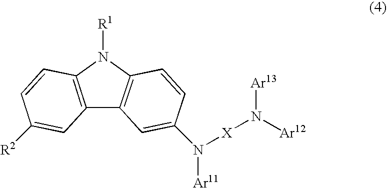
(In the formula, Ar¹¹ is an aryl group having 7 to 25 carbon atoms or a heteroaryl group having 7 to 25 carbon atoms. Ar¹² and Ar¹³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms. R¹ is any one of hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms. R² is any one of hydrogen, an alkyl group having 1 to 6 carbon atoms, or a substituent represented by General Formula 5. 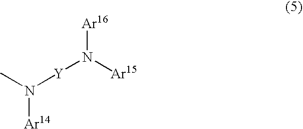
In General Formula 5, Ar¹⁴ is either an aryl group having 7 to 25 carbon atoms or a heteroaryl group having 7 to 25 carbon atoms. Ar¹⁵ and Ar¹⁶ may be identical or different, and are individually either an aryl group having 6 to 25 carbon atoms or a heteroaryl group having 5 to 9 carbon atoms. Y is either a bivalent aromatic hydrocarbon group having 6 to 25 carbon atoms or a bivalent heterocyclic group having 5 to 10 carbon atoms.)

One feature of the present invention is a material for a light emitting element, represented by General Formula 6. 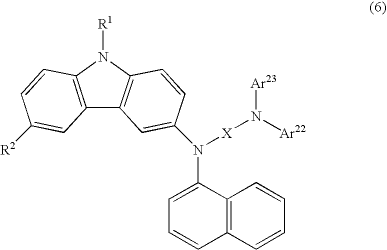
(In the formula, Ar²² and Ar²³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms. R¹ is any one of hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms. R² is any one of hydrogen, an alkyl group having 1 to 6 carbon atoms, or a substituent represented by General Formula 7. 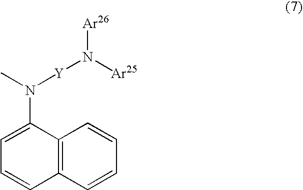
In General Formula 7, Ar²⁵ and Ar²⁶ may be identical or different, and are individually either an aryl group having 6 to 25 carbon atoms or a heteroaryl group having 5 to 9 carbon atoms. Y is either a bivalent aromatic hydrocarbon group having 6 to 25 carbon atoms or a bivalent heterocyclic group having 5 to 10 carbon atoms.)

One feature of the present invention is a material for a light emitting element, represented by General Formula 8. 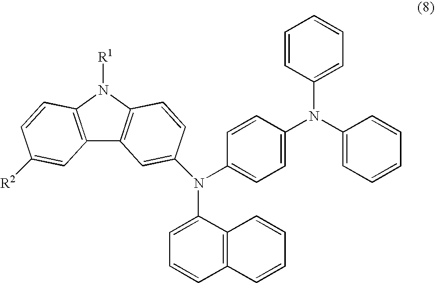
(In the formula, R¹ is any one of hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms. R² is any one of hydrogen, an alkyl group having 1 to 6 carbon atoms, or a substituent represented by Structural Formula 9.)

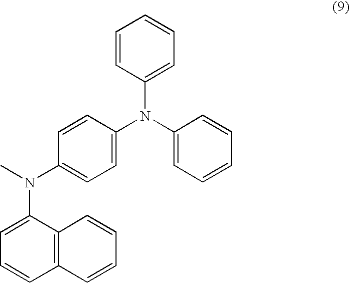

One feature of the present invention is a light emitting element which includes a layer containing a light emitting substance between a pair of electrodes, in which the layer containing the light emitting substance contains the above-described material for a light emitting element.

Another feature of the present invention is a light emitting element which includes a first electrode, a second electrode, and a layer containing a light emitting substance between the first electrode and the second electrode, in which the layer containing the light emitting substance includes a light emitting layer and a layer containing the above-described material for a light emitting element provided on a first electrode side of the light emitting layer, and the light emitting substance emits light when a voltage is applied such that a potential of the first electrode is higher than that of the second electrode.

One feature of the present invention is a light emitting element which includes a layer containing a light emitting substance between a pair of electrodes and a light emitting layer included in the layer containing the light emitting substance, in which the light emitting layer contains the above-described material for a light emitting element.

One feature of the present invention is a light emitting element which includes a layer containing a light emitting substance between a pair of electrodes, in which the light emitting substance is the above-described material for a light emitting element.

Moreover, the present invention includes a light emitting device having the above-described light emitting element. The light emitting device in this specification includes an image display device, a light emitting device or a light source (including a lighting device). In addition, the light emitting device of the present invention includes a module in which a panel formed with a light emitting element is attached using a connector such as an FPC (flexible printed circuit), a TAB (tape automated bonding) tape or a TCP (tape carrier package); a module in which a printed wiring board is provided on the tip of a TAB tape or a TCP; and a module in which an IC (integrated circuit) is directly mounted on a light emitting element by COG (chip on glass).

Further, the present invention includes an electronic device using a light emitting element of the present invention in a display portion. Accordingly, one feature of the electronic device of the present invention is to include a display portion, in which the display portion includes the above-described light emitting element and a control means which controls light emission of the light emitting element.

A tertiary arylamine compound obtained by using the secondary arylamine compound of the present invention has an excellent hole transporting property and an excellent hole injecting property. In addition, the tertiary arylamine compound is easily oxidized and the tertiary arylamine compound in an oxidation state is stable, and returns to a neutral state by subsequent reduction. In other words, the tertiary arylamine compound obtained by using the secondary arylamine compound of the present invention is stable even when an oxidation state and a neutral state are repeated by an oxidation reaction and a reduction reaction subsequent to the oxidation.

A material for a light emitting element, which is the tertiary arylamine compound obtained by using the secondary amine compound of the present invention, is stable even when an oxidation state and a neutral state are repeated by an oxidation reaction and a reduction reaction subsequent to the oxidation. This means that the tertiary arylamine compound has resistance to repeated oxidation reactions. Therefore, by using the material for a light emitting element of the present invention, a light emitting element and an electronic device which have high reliability and long life can be obtained.

### BRIEF DESCRIPTION OF DRAWINGS

In the accompanying drawings:

FIGS. 1A to 1C show light emitting elements of the present invention;

FIG. 2 shows a light emitting element of the present invention;

FIGS. 3A and 3B show a light emitting device of the present invention;

FIG. 4 shows a light emitting device of the present invention;

FIGS. 5A to 5D show electronic devices of the present invention;

FIG. 6 shows an electronic device of the present invention;

FIG. 7 is a ¹H-NMR chart of N-(4-diphenylaminophenyl)-N-(1-naphthyl)amine;

FIG. 8 is a ¹H-NMR chart of N-(4-diphenylaminophenyl)-N-(1-naphthyl)amine;

FIG. 9 is a ¹³C-NMR chart of N-(4-diphenylaminophenyl)-N-(1-naphthyl)amine;

FIG. 10 is a ¹H-NMR chart of 3-[N-(4-diphenylaminophenyl)-N-(1-naphthyl)amino]-9-phenylcarbazole;

FIG. 11 is a ¹H-NMR chart of 3-[N-(4-diphenylaminophenyl)-N-(1-naphthyl)amino]-9-phenylcarbazole;

FIG. 12 shows absorption spectra of 3-[N-(4-diphenylaminophenyl)-N-(1-naphthyl)amino]-9-phenylcarbazole;

FIG. 13 shows emission spectra of 3-[N-(4-diphenylaminophenyl)-N-(1-naphthyl)amino]-9-phenylcarbazole;

FIG. 14 shows a CV measurement result of 3-[N-(4-diphenylaminophenyl)-N-(1-naphthyl)amino]-9-phenylcarbazole;

FIG. 15 is a ¹H-NMR chart of 3,6-bis[N-(4-diphenylaminophenyl)-N-(1-naphthyl)amino]-9-phenylcarbazole;

FIG. 16 is a ¹H-NMR chart of 3,6-bis[N-(4-diphenylaminophenyl)-N-(1-naphthyl)amino]-9-phenylcarbazole;

FIG. 17 shows absorption spectra of 3,6-bis[N-(4-diphenylaminophenyl)-N-(1-naphthyl)amino]-9-phenylcarbazole;

FIG. 18 shows emission spectra of 3,6-bis[N-(4-diphenylaminophenyl)-N-(1-naphthyl)amino]-9-phenylcarbazole;

FIG. 19 shows a CV measurement result of 3,6-bis[N-(4-diphenylaminophenyl)-N-(1-naphthyl)amino]-9-phenylcarbazole;

FIG. 20 shows a light emitting element of Example 4;

FIG. 21 shows luminance-voltage characteristics of a light emitting element of Example 4;

FIG. 22 shows luminance-current efficiency characteristics of a light emitting element of Example 4; and

FIG. 23 shows an emission spectrum of a light emitting element of Example 4.

### BEST MODE FOR CARRYING OUT THE INVENTION

Embodiment modes and examples of the present invention will be described in detail with reference to the drawings. Note that it is easily understood by those skilled in the art that the invention is not limited to the following descriptions, and various changes may be made in forms and details without departing from the spirit and the scope of the invention. Therefore, the invention should not be limited to the descriptions of the embodiment modes and examples below.

### EMBODIMENT MODE 1

A secondary arylamine compound and a synthetic method thereof according to the present invention will be described.

The secondary arylamine compound of the present invention is represented by General Formula 1. 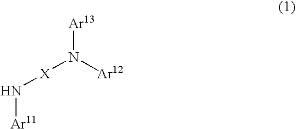
(In the formula, Ar¹¹ is an aryl group having 7 to 25 carbon atoms or a heteroaryl group having 7 to 25 carbon atoms. Ar¹² and Ar¹³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms.)

As the aryl group having 6 to 25 carbon atoms, specifically, a phenyl group, a 4-biphenylyl group, a 1-naphthyl group, a 2-naphthyl group, a 9-anthryl group, a 9-phenanthryl group, a 1-pyrenyl group, a 9,9′-dimethyl-2-fluorenyl group, a 9,9′-diphenyl-2-fluorenyl group, a spiro-9,9′-bifluorene-2-yl group, or the like can be used. Further, an aryl group having a substituent such as an m-tolyl group, a p-tolyl group, a 2-fluorophenyl group, a 3-fluorophenyl group, or a 4-fluorophenyl group may be used.

As the heteroaryl group having 5 to 9 carbon atoms, specifically, a 2-pyridyl group, a 8-quinolyl group, a 3-quinolyl group, or the like can be used.

As the aryl group having 7 to 25 carbon atoms, specifically, a 4-biphenylyl group, a 1-naphthyl group, a 2-naphthyl group, a 9-anthryl group, a 9-phenanthryl group, a 1-pyrenyl group, a 9,9′-dimethyl-2-fluorenyl group, a 9,9′-diphenyl-2-fluorenyl group, a spiro-9,9′-bifluorene-2-yl group, or the like can be used. Further, an aryl group having a substituent such as an m-tolyl group, a p-tolyl group, a 2-fluorophenyl group, a 3-fluorophenyl group, or a 4-fluorophenyl group may be used.

As the heteroaryl group having 7 to 9 carbon atoms, specifically, a 8-quinolyl group, a 3-quinolyl group, or the like can be used.

Further, as the bivalent aromatic hydrocarbon group having 6 to 25 carbon atoms, specifically, bivalent aromatic hydrocarbon groups represented by Structural Formulas 11 to 23 can be used.

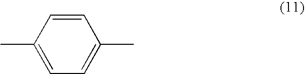

Further, as the bivalent heterocyclic group having 5 to 10 carbon atoms, specifically, bivalent heterocyclic groups represented by Structural Formulas 24 to 29 can be used.

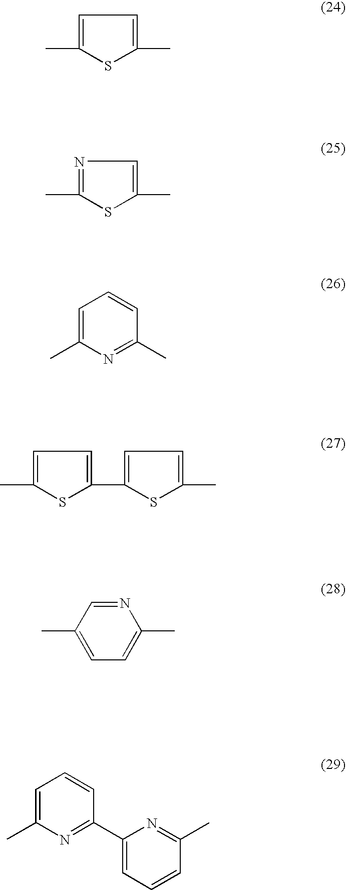

It is preferable to use a secondary arylamine compound represented by General Formula 2 of the secondary arylamine compounds which are represented by General Formula 1. 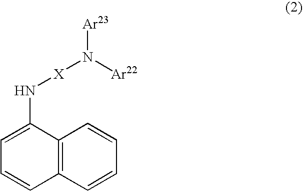
(In the formula, Ar²² and Ar²³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms.)

As a specific mode of the secondary arylamine compound represented by General Formula 1, secondary arylamine compounds represented by Structural Formulas 31 to 54 can be provided.

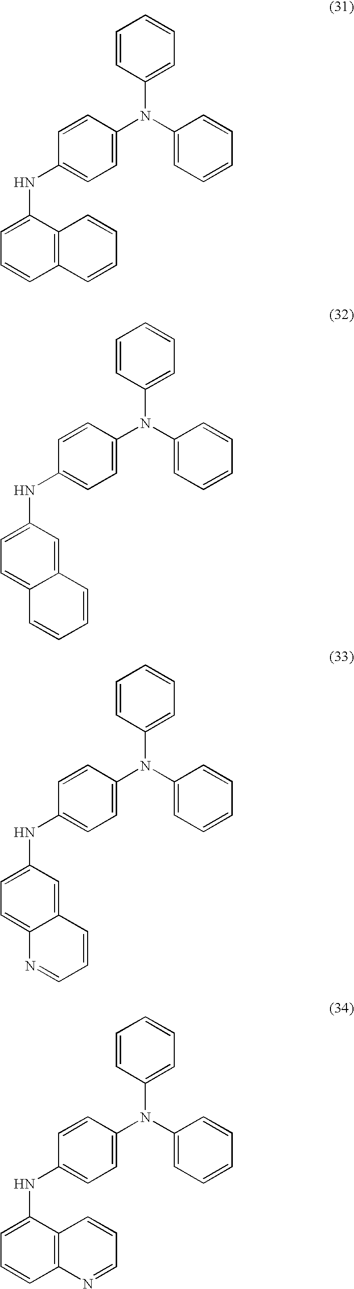

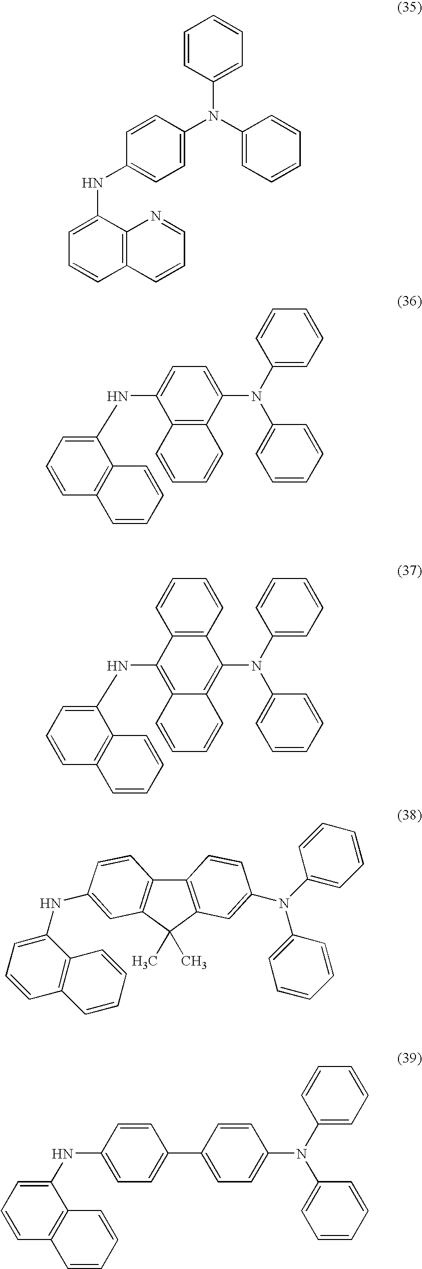

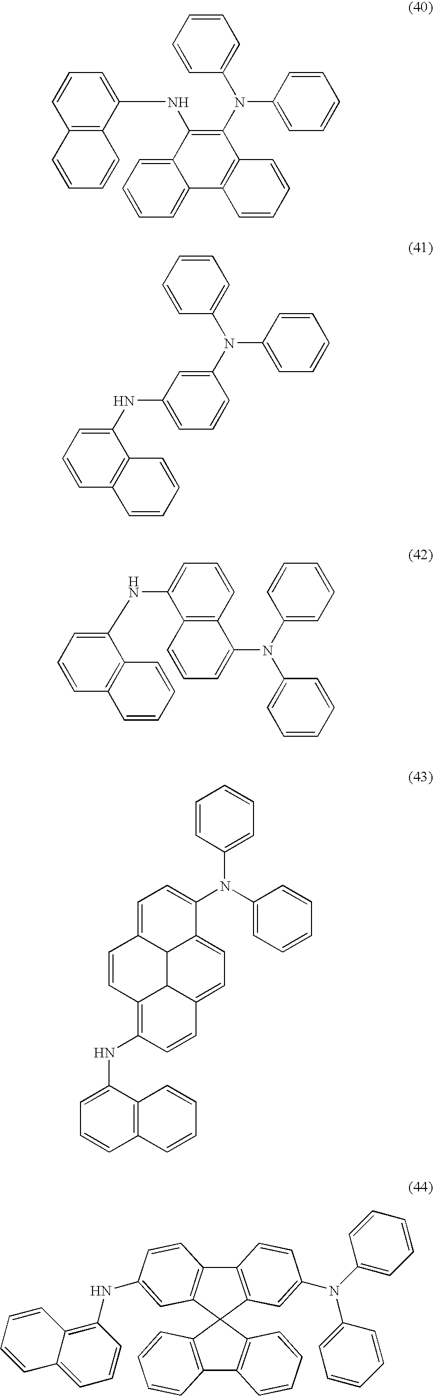

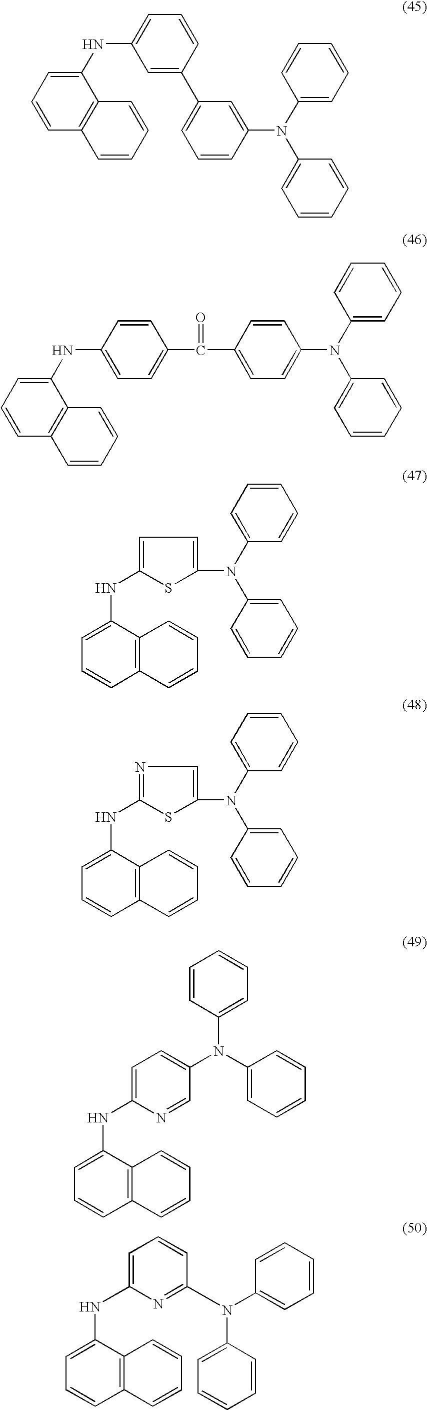

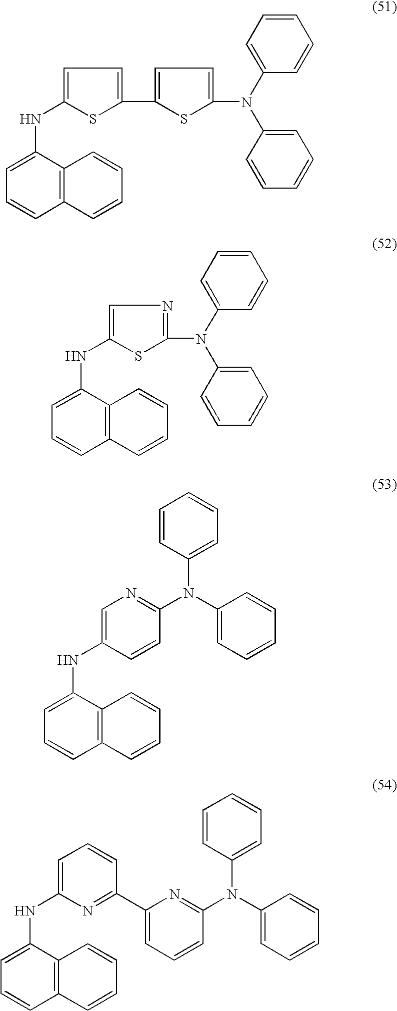

In particular, a secondary arylamine compound represented by Structural Formula 31 is preferable since it can be easily synthesized.

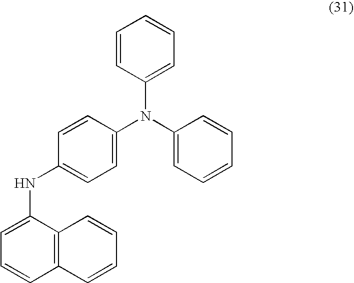

A secondary arylamine compound of the present invention can be synthesized by a method shown in Synthetic Scheme (A-1).

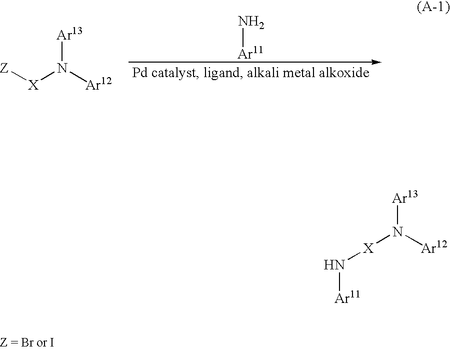

A reaction of primary arylamine and a tertiary arylamine halide is performed by using a Pd catalyst including (t-Bu)₃P as a ligand. For example, by mixing Pd(dba)₂ and (t-Bu)₃P, the (t-Bu)₃P is coordinated to Pd. As well as Pd(dba)₂, a Pd complex to which a ligand having a smaller coordination power than (t-Bu)₃P is coordinated, may also be used. Specifically, Pd(dba)₂, Pd(OAc)₂, or the like can be used. Preferably, Pd(dba)₂ is used. As a ligand, as well as (t-Bu)₃P, DPPF can be used. The reaction temperature is preferably in the range of from room temperature to 130° C. In the case of heating to 130° C. or more, the Pd catalyst is decomposed and cannot serve as a catalyst. The reaction temperature is preferably set from 60 to 110° C. since reaction is easily controlled and yield is improved. The term “dba” refers to trans, trans-dibenzylideneacetone. The term “DPPF” refers to 1,1-bis(diphenylphosphino)ferrocene. As a solvent, dehydrated toluene, xylene, or the like can be used. As a base, alkali metal alcoxide or the like such as t-BuONa can be used.

A tertiary arylamine compound formed by using the above-described secondary arylamine compound of the present invention has an excellent hole transporting property and an excellent hole injecting property. In addition, the tertiary arylamine compound is easily oxidized and the tertiary arylamine compound in an oxidation state is stable, and returns to a neutral state by subsequent reduction. In other words, the tertiary arylamine compound formed by using the secondary amine compound of the present invention is stable even when an oxidation state and a neutral state are repeated by an oxidation reaction and a reduction reaction subsequent to the oxidation. This means that the tertiary arylamine compound has resistance to repeated oxidation reactions.

By depositing the tertiary arylamine compound formed by using the secondary arylamine compound of the present invention, an amorphous film can be easily obtained. Therefore, it can be favorably used in a light emitting element.

### EMBODIMENT MODE 2

Embodiment Mode 2 will describe a material for a light emitting element, which can be obtained by using a secondary arylamine compound of the present invention.

As one mode of the material for a light emitting element using the secondary arylamine compound shown in Embodiment Mode 1, a carbazole derivative represented by General Formula 4 can be used. 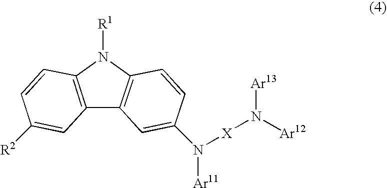
(In the formula, Ar¹¹ is an aryl group having 7 to 25 carbon atoms or a heteroaryl group having 7 to 25 carbon atoms. Ar¹² and Ar¹³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms. R¹ is any one of hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms. R² is any one of hydrogen, an alkyl group having 1 to 6 carbon atoms, or a substituent represented by General Formula 5. 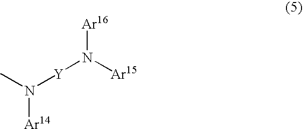
(In General Formula 5, Ar¹⁴ is either an aryl group having 7 to 25 carbon atoms or a heteroaryl group having 7 to 25 carbon atoms. Ar¹⁵ and Ar¹⁶ may be identical or different, and are individually either an aryl group having 6 to 25 carbon atoms or a heteroaryl group having 5 to 9 carbon atoms. Y is either a bivalent aromatic hydrocarbon group having 6 to 25 carbon atoms or a bivalent heterocyclic group having 5 to 10 carbon atoms.)

As the aryl group having 6 to 25 carbon atoms, specifically, a phenyl group, a 4-biphenylyl group, a 1-naphthyl group, a 2-naphthyl group, a 9-anthryl group, a 9-phenanthryl group, a 1-pyrenyl group, a 9,9′-dimethyl-2-fluorenyl group, a 9,9′-diphenyl-2-fluorenyl group, a spiro-9,9′-bifluorene-2-yl group, or the like can be used. Further, an aryl group having a substituent such as an m-tolyl group, a p-tolyl group, a 2-fluorophenyl group, a 3-fluorophenyl group, or a 4-fluorophenyl group may be used.

As the heteroaryl group having 5 to 9 carbon atoms, specifically, a 2-pyridyl group, a 8-quinolyl group, a 3-quinolyl group, or the like can be used.

As the aryl group having 7 to 25 carbon atoms, specifically, a 4-biphenylyl group, a 1-naphthyl group, a 2-naphthyl group, a 9-anthryl group, a 9-phenanthryl group, a 1-pyrenyl group, a 9,9′-dimethyl-2-fluorenyl group, a 9,9′-diphenyl-2-fluorenyl group, a spiro-9,9′-bifluorene-2-yl group, or the like can be used. Further, an aryl group having a substituent such as an m-tolyl group, a p-tolyl group, a 2-fluorophenyl group, a 3-fluorophenyl group, or a 4-fluorophenyl group may be used.

As the heteroaryl group having 7 to 9 carbon atoms, specifically, a 8-quinolyl group, a 3-quinolyl group, or the like can be used.

Further, as the bivalent aromatic hydrocarbon group having 6 to 25 carbon atoms, specifically, bivalent aromatic hydrocarbon groups represented by Structural Formulas 11 to 23 can be used.

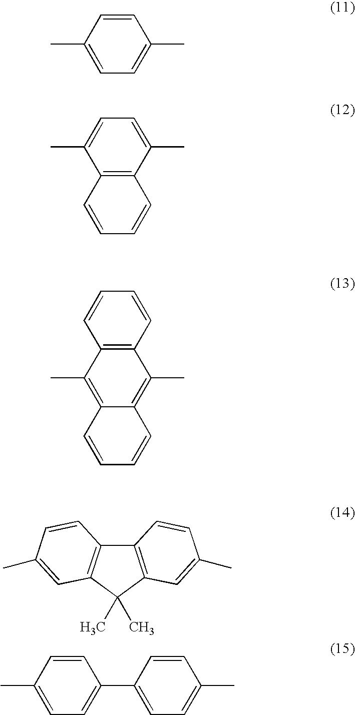

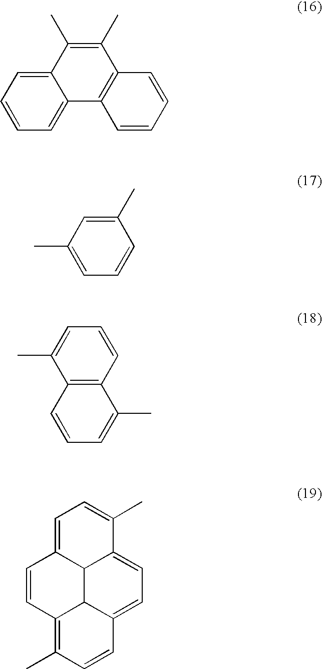

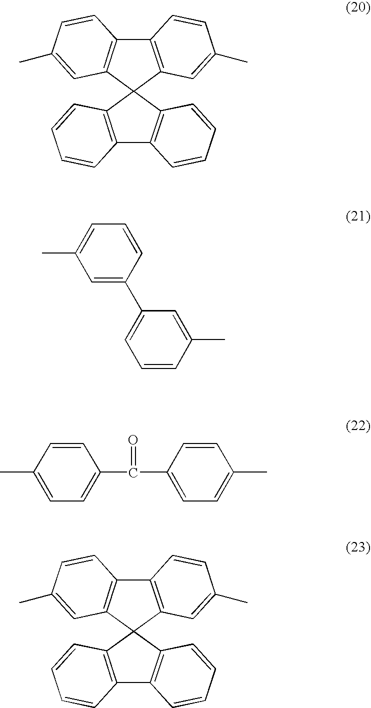

Further, as the bivalent heterocyclic group having 5 to 10 carbon atoms, specifically, bivalent heterocyclic groups represented by Structural Formulas 24 to 29 can be used.

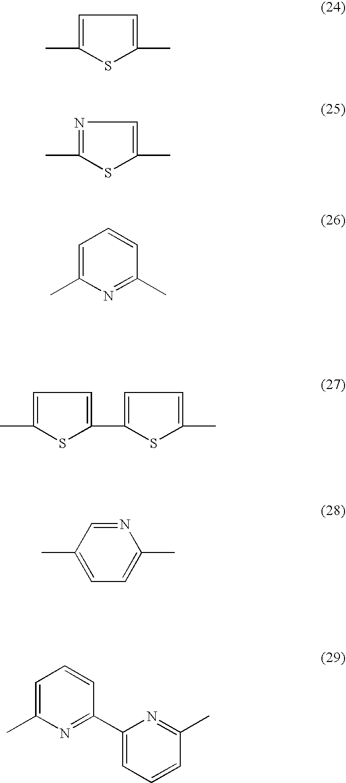

In the above structure, it is preferable that R¹ be any one of a methyl group, an ethyl group, a tert-butyl group, or a phenyl group.

Further, in the above structure, it is preferable that R² be either hydrogen or a tert-butyl group. Alternatively, it is preferable that R² have a structure shown in General Formula 5.

As another mode of the material for a light emitting element using the secondary arylamine compound described in Embodiment Mode 1, a carbazole derivative represented by General Formula 6 can be used. 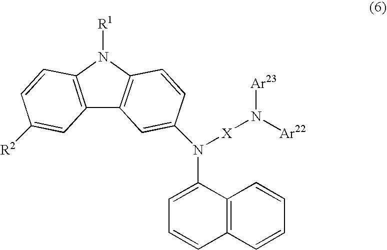
(In the formula, Ar²² and Ar²³ may be identical or different, and are individually either an aryl group having 6 to 25 carbon atoms or a heteroaryl group having 5 to 9 carbon atoms. X is either a bivalent aromatic hydrocarbon group having 6 to 25 carbon atoms or a bivalent heterocyclic group having 5 to 10 carbon atoms. R¹ is any one of hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms. R² is any one of hydrogen, an alkyl group having 1 to 6 carbon atoms, or a substituent represented by General Formula 7. 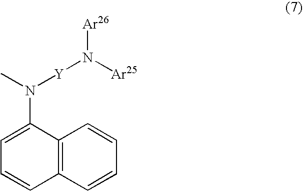
(In General Formula 7, Ar²⁵ and Ar²⁶ may be identical or different, and are individually either an aryl group having 6 to 25 carbon atoms or a heteroaryl group having 5 to 9 carbon atoms. Y is either a bivalent aromatic hydrocarbon group having 6 to 25 carbon atoms or a bivalent heterocyclic group having 5 to 10 carbon atoms.)

In the above structure, it is preferable that R¹ be any one of a methyl group, an ethyl group, a tert-butyl group, or a phenyl group.

In the above structure, it is preferable that R² be either hydrogen or a tert-butyl group. Alternatively, it is preferable that R² have a structure shown in General Formula 7.

As another mode of the material for a light emitting element using the secondary arylamine compound described in Embodiment Mode 1, a carbazole derivative represented by General Formula 8 can be used. 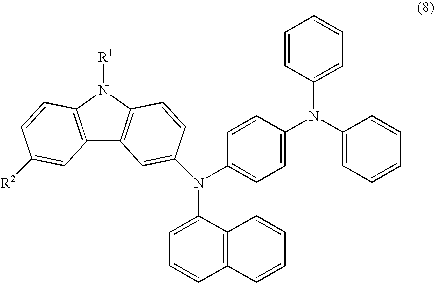
(In the formula, R¹ is any one of hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms. R² is any one of hydrogen, an alkyl group having 1 to 6 carbon atoms, or a substituent represented by Structural Formula 9.)

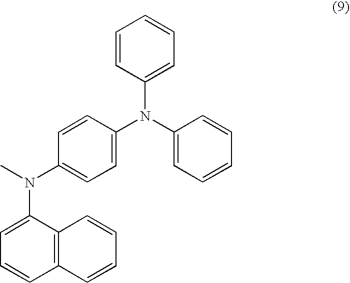

In the above structure, it is preferable that R¹ be any one of a methyl group, an ethyl group, a tert-butyl group, or a phenyl group.

In the above structure, it is preferable that R² be either hydrogen or a tert-butyl group. Alternatively, it is preferable that R² have a structure shown in Structural Formula 9.

A material for a light emitting element, which is a tertiary arylamine compound obtained by using a secondary arylamine compound of the present invention has an excellent hole transporting property and a hole injecting property. Therefore, a light emitting element whose driving voltage is reduced can be obtained.

A material for a light emitting element, which is the tertiary arylamine compound obtained by using a secondary arylamine compound of the present invention, is easily oxidized and stable in an oxidation state, and returns to a neutral state by subsequent reduction. In other words, the material for a light emitting element which is the tertiary arylamine compound obtained by using a secondary arylamine compound of the present invention is stable even when an oxidation state and a neutral state are repeated by an oxidation reaction and a reduction reaction subsequent to the oxidation. This means that the tertiary arylamine compound has resistance to repeated oxidation reactions. By depositing the material for a light emitting element which is the tertiary arylamine compound obtained by using a secondary arylamine compound of the present invention, an amorphous film can be obtained. Therefore, a long-life light emitting element can be obtained.

### EMBODIMENT MODE 3

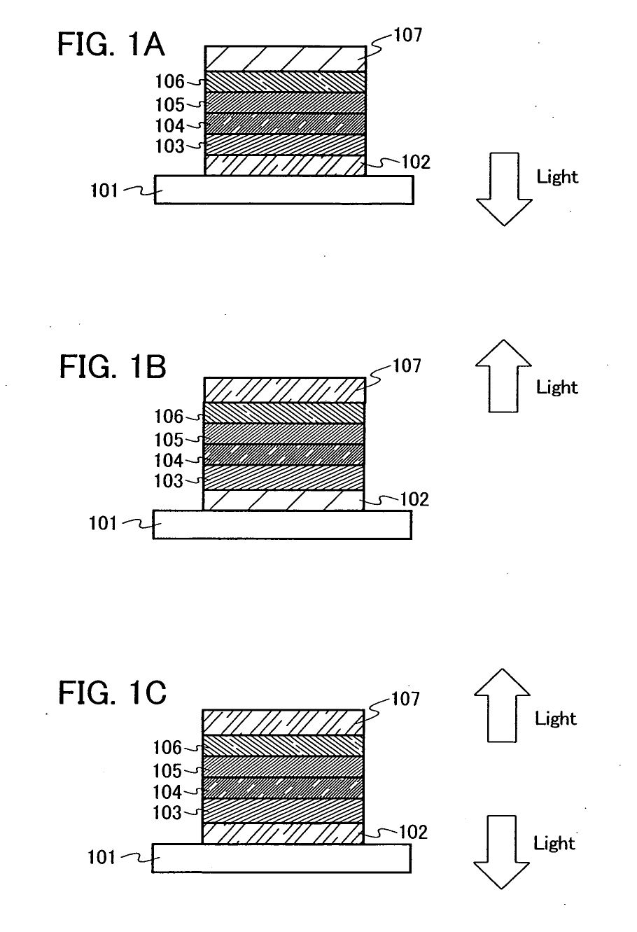

One mode of a light emitting element which uses the material for a light emitting element obtained by using a secondary arylamine compound of the present invention will be described with reference to FIG. 1A.

The light emitting element of the present invention includes a plurality of layers between a pair of electrodes. The plurality of layers are stacked layers formed by combining layers containing a substance having a high carrier injecting property or a high carrier transporting property, so that a light emitting region is formed in a place which is away from the electrodes, that is, so as to perform recombination of carriers in a portion which is away from the electrodes.

In this embodiment mode, the light emitting element includes a first electrode 102; a first layer 103, a second layer 104, a third layer 105, and a forth layer 106 which are sequentially stacked over the first electrode 102; and a second electrode 107 further provided thereover. The following description is made of the condition that the first electrode 102 serves as an anode and the second electrode 107 serves as a cathode.

A substrate 101 is used as a support medium of the light emitting element. As the substrate 101, glass, plastic, or the like can be used for example. Note that another material may be used as long as the material functions as a support medium in the manufacturing process of the light emitting element.

As the first electrode 102, a metal, an alloy, a conductive compound, a mixture thereof, or the like having a high work function (specifically, of 4.0 eV or more) is preferably used. Specifically, indium tin oxide (ITO), indium tin oxide containing silicon, indium zinc oxide (IZO) in which indium oxide is mixed with 2 to 20 wt % of zinc oxide (ZnO), indium oxide (IWZO) containing 0.5 to 5 wt % of tungsten oxide and 0.1 to 1 wt % of zinc oxide, or the like can be used, for example. Such a conductive metal oxide film is usually formed by a sputtering method, but may be formed by applying a sol-gel method or the like. Further, gold (Au), platinum (Pt), nickel (Ni), tungsten (W), chromium (Cr), molybdenum (Mo), iron (Fe), cobalt (Co), copper (Cu), palladium (Pd), a nitride of a metal material (for example, titanium nitride: TiN), or the like can be used.

The first layer 103 is a layer containing a high hole injecting property, and molybdenum oxide (MoOₓ), vanadium oxide (VOₓ), ruthenium oxide (RuOₓ), tungsten oxide (WOₓ), manganese oxide (MnOₓ), or the like can be used. Alternatively, the first layer 103 can be formed by using a phthalocyanine-based compound such as phthalocyanine (H₂Pc) or copper phthalocyanine (CuPc), a high molecular material such as poly(ethylene dioxythiophene)/poly(styrenesulfonic acid) (PEDOT/PSS), or the like. In addition, since the material for a light emitting element, which is the tertiary arylamine compound obtained by using a secondary arylamine compound of the present invention has an excellent hole injecting property, it can be used for the first layer 103.

A composite material containing an organic compound and an inorganic compound may also be used for the first layer 103. In particular, a composite material containing an organic compound and an inorganic compound which has an electron accepting property to the organic compound, has an excellent hole injecting property and an excellent hole transporting property, because electrons are transferred between the organic compound and the inorganic compound to increase a carrier density. In this case, it is preferable to use a material having an excellent hole transporting property as the organic compound. Specifically, an aromatic amine-based organic compound or a carbazole-based organic compound is preferable. Since the material for a light emitting element, which is the tertiary arylamine compound obtained by using a secondary arylamine compound of the present invention, has an excellent hole transporting property, it can be used as the composite material by being combined with an inorganic compound, so as to form the first layer 103. Further, as the organic compound, aromatic hydrocarbon may be used. As the inorganic compound, a substance having an electron accepting property to the organic compound is preferable, and an oxide of a transition metal is specifically preferable. For example, a metal oxide such as titanium oxide (TiOₓ), vanadium oxide (VOₓ), molybdenum oxide (MoOₓ), tungsten oxide (WOₓ), rhenium oxide (ReOₓ), ruthenium oxide (RuOₓ), chromium oxide (CrOₓ), zirconium oxide (ZrOₓ), hafnium oxide (HfOₓ), tantalum oxide (TaOₓ), silver oxide (AgOₓ), or manganese oxide (MnOₓ) can be used. In the case of using the composite material containing an organic compound and an inorganic compound for the first layer 103, an ohmic contact with the first electrode 102 can be made; therefore, the material for the first electrode 102 can be selected regardless of the work function.

As a substance for forming the second layer 104, a substance having a high hole transporting property, specifically, an aromatic amine-based (i.e., a material having a benzene ring-nitrogen bond) compound is preferable. As the material which is widely used, a starburst aromatic amine compound such as 4,4′-bis[N-(3-methylphenyl)-N-phenylamino]biphenyl; a derivative thereof: 4,4′-bis[N-(1-naphthyl)-N-phenylamino]biphenyl (hereinafter referred to as NPB); 4,4′,4″-tris(N,N-diphenyl-amino)triphenylamine; or 4,4′,4″-tris[N-(3-methylphenyl)-N-phenylamino]triphenylamine can be used. The substances noted herein are substances each having a hole mobility of 10⁻⁶ cm²/Vs or more mainly. However, other substances may be used if the substances have a hole transporting property higher than an electron transporting property. In addition, since the material for a light emitting element, which is the tertiary arylamine compound obtained by using a secondary arylamine compound of the present invention has an excellent hole transporting property, it can be used for the second layer 104. As the second layer 104, a mixed layer of the above-described substances or a stacked layer including two or more layers may be employed as well as a single layer.

The third layer 105 has a layer containing a light emitting substance. The light emitting substance is not particularly limited and various kinds of substances can be used. As the light emitting substance, the following can be used: a coumarin derivative such as coumarin 6 or coumarin 545T; a quinacridone derivative such as N,N′-dimethyl quinacridone or N,N′-diphenyl quinacridone; an acridone derivative such as N-phenylacridone or N-methyl acridone; a condensed aromatic compound such as 2-t-butyl-9,10-di(2-naphthyl)anthracene (t-BuDNA), 9,10-diphenylanthracene, 2,5,8,11-tetra-t-butylperylene, or rubrene; a pyran derivative such as 4-dicyanomethylene-2-[p-(dimethylamino)styryl]6-methyl-4H-pyran, an amine derivative such as 4-(2,2-diphenylvinyl)triphenylamine, or the like. As a phosphorescent substance, an iridium complex such as bis{2-(4-tolyl)pyridinato}(acetylacetonato)iridium(III), bis{2-(2′-benzothienyl)pyridinato}(acetylacetonato)iridium(III), or bis{2-(4,6-difluorophenyl)pyridinato]picolinatoiridium(III); a platinum complex such as 2,3,7,8,12,13,17,18-octaethyl-21H,23H-porphyrin-platinum complex, a rare-earth complex such as 4,7-diphenyl-1,10-phenanthroline-tris(2-thiophenyltrifluoroacetonato)europium(III), or the like can be used.

The material for a light emitting element of the present invention can be used as a light emitting substance. A secondary arylamine compound of the present invention is stable even when an oxidation state and a neutral state are repeated by an oxidation reaction and a reduction reaction subsequent to the oxidation. By chemically binding the secondary arylamine compound of the present invention to a substituent which is stable even when an reduction state and a neutral state are repeated by a reduction reaction and an oxidation reaction subsequent thereto, a light emitting substance which is stable in repeated oxidation-reduction reactions can be obtained. As the substituent which is stable even when a reduction reaction and an oxidation reaction subsequent thereto are repeated, a substituent including diphenylanthracene, a substituent including stilbene, or the like can be used.

As a material for dispersing the light emitting substance, various kinds of substances can be used. Specifically, a substance having a higher LUMO level and a lower HOMO level than the light emitting substance can be used. As the material for dispersing the light emitting substance, plural kinds of materials can be used. For example, in order to suppress crystallization, a substance such as rubrene which suppresses crystallization, may be further added. In addition, in order to more efficiently perform energy transfer to the light emitting substance, 4,4′-bis[N-(1-naphthyl)-N-phenylamino]biphenyl (NPB), tris(8-quinolinolato)aluminum (Alq), or the like may be further added.

Note that as the material for dispersing the light emitting substance, the material for a light emitting element of the present invention can be used.

The fourth layer 106 is formed by using a substance having a high electron transporting property, e.g., a metal complex having a quinoline skeleton or a benzoquinoline skeleton such as tris(8-quinolinolato)aluminum (Alq), tris(4-methyl-8-quinolinolato)aluminum (Almq₃), bis(10-hydroxybenzo[h]-quinolinato)beryllium (BeBq₂), or bis(2-methyl-8-quinolinolato)(4-phenylphenolato)aluminum (BAlq). Alternatively, a metal complex or the like having an oxazole ligand or a thiazole ligand, such as bis[2-(2′-hydroxyphenyl)benzoxazolato]zinc (Zn(BOX)₂) or bis[2-(2′-hydroxyphenyl)benzothiazolato]zinc (Zn(BTZ)₂) can also be used. Other than the metal complex, 2-(4-biphenylyl)-5-(4-tert-buthylphenyl)-1,3,4-oxadiazole (PBD), 1,3-bis[5-(p-tert-buthylphenyl)-1,3,4-oxadiazol-2-yl]benzene (OXD-7), 3-(4-tert-butylphenyl)-4-phenyl-5-(4-biphenylyl)-1,2,4-triazole (TAZ), 3-(4-tert-butylphenyl)-4-(4-ethylphenyl)-5-(4-biphenylyl)-1,2,4-triazole (p-EtTAZ), bathophenanthroline (BPhen), bathocuproin (BCP), or the like can be employed. The substances noted herein are substances each having an electron mobility of 10⁻⁶ cm²/Vs or more mainly. However, other substances may be used for the fourth layer 106 if the substances have an electron transporting property higher than a hole transporting property. As the fourth layer 106, a stacked layer including two or more layers each containing the above-described substance may be employed as well as a single layer.

As a substance for forming the second electrode 107, a metal, an alloy, a conductive compound, a mixture thereof, or the like having a low work function (specifically, of 3.8 eV or less) can be used. As a specific example of such a cathode material, an element belonging to group 1 or 2 in the periodic table, that is, an alkali metal such as lithium (Li) or cesium (Cs); an alkaline earth metal such as magnesium (Mg), calcium (Ca), or strontium (Sr); an alloy containing the element belonging to group 1 or 2 (MgAg, AlLi); a rare-earth metal such as europium (Eu) or ytterbium (Yb); an alloy thereof; or the like can be used. However, various conductive materials such as Al, Ag, ITO, and ITO containing silicon, regardless of the work function, can be used for the second electrode 107, by providing a layer having a function of promoting electron injection between the second electrode 107 and the light emitting layer.

For the layer having a function of promoting electron injection, a compound of an alkali metal or an alkaline earth metal such as lithium fluoride (LiF), cesium fluoride (CsF), or calcium fluoride (CaF₂) can be used. Alternatively, a layer containing an electron transporting substance, into which an alkali metal or an alkaline earth metal is added, for example, Alq into which magnesium (Mg) or lithium (Li) is added or the like, can be used.

In forming the first layer 103, the second layer 104, the third layer 105, and the fourth layer 106, various kinds of forming methods can be employed, such as an inkjet method or a spin coating method, as well as an evaporation method. It is to be noted that differing film forming methods can be used for each layers and the electrodes.

A current flows due to a potential difference generated between the first electrode 102 and the second electrode 107, and holes and electrons are recombined in the third layer 105 which is a layer containing a highly light emitting substance; accordingly, the light emitting element of the present invention having the above-described structure emits light. In other words, a structure where a light emitting region is formed in the third layer 105 is obtained.

The light is extracted through either or both of the first electrode 102 and the second electrode 107. Therefore, either or both of the first electrode 102 and the second electrode 107 is formed with a light-transmitting substance. In the case where only the first electrode 102 is formed with a light-transmitting substance, light is extracted from a substrate side through the first electrode 102, as shown in FIG. 1A. In the case where only the second electrode 107 is formed with a light-transmitting substance, light is extracted from an opposite side to the substrate through the second electrode 107, as shown in FIG. 1B. Further, when both of the first electrode 102 and the second electrode 107 are formed with light-transmitting substances, light is extracted from both the substrate side and the opposite side to the substrate through the first electrode 102 and the second electrode 107, as shown in FIG 1C.

Note that the structure of layers provided between the first electrode 102 and the second electrode 107 is not limited to the above-described structure. Other structures may be employed as long as a light emitting region for recombining holes and electrons is provided in a place which is away from the first electrode 102 and the second electrode 107 for the purpose of suppressing quenching caused by adjacence of the light emitting region and a metal.

That is, the stacked structure of layers is not particularly limited, and a layer containing a substance having a high electron transporting property, a substance having a high hole transporting property, a substance having a high electron injecting property, a substance having a high hole injecting property, a bipolar substance (a substance having high electron or hole transporting property), a hole blocking material, or the like, may be freely combined with the material for a light emitting element, which is the tertiary arylamine compound obtained by using the secondary arylamine compound of the present invention.

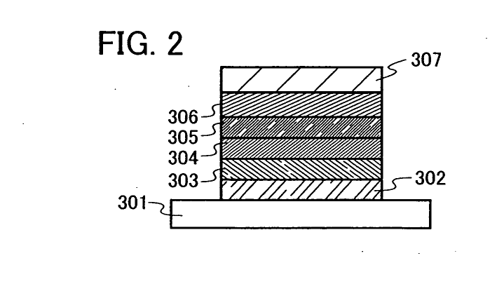

A light emitting element shown in FIG. 2 has a structure in which a first layer 303 containing a substance having a high electron transporting property, a second layer 304 containing a light emitting substance, a third layer 305 containing a substance having a high hole transporting property, a fourth layer 306 containing a substance having a high hole injecting property, and a second electrode 307 serving as an anode, are stacked in order over a first electrode 302 serving as a cathode. Note herein that reference numeral 301 denotes a substrate.

In this embodiment mode, the light emitting element is manufactured over a substrate formed of glass, plastic, or the like. By manufacturing a plurality of light emitting elements over one substrate, a passive light emitting device can be manufactured. In addition, a thin film transistor (TFT) may be, for example, formed over a substrate containing glass, plastic, or the like, and a light emitting element may be manufactured over an electrode electrically connected to the TFT. Thus, an active-matrix light emitting device which controls driving of the light emitting element by using the TFT can be manufactured. The structure of the TFT is not particularly limited, and a staggered TFT or an inversely staggered TFT may be used. A driver circuit formed over a TFT array substrate may be formed with either or both of an n-type TFT and a p-type TFT.

The material for a light emitting element, which is the tertiary arylamine compound obtained by using a secondary arylamine compound of the present invention, has an excellent hole transporting property and a hole injecting property. Accordingly, a light emitting element having favorable characteristics can be obtained by using the material, for the light emitting element. Specifically, a light emitting element whose driving voltage is low can be obtained.

The material for a light emitting element, which is the tertiary arylamine compound obtained by using a secondary arylamine compound of the present invention, is easily oxidized and stable in an oxidation state, and returns to a neutral state by subsequent reduction. In other words, the material for a light emitting element which is the tertiary arylamine compound obtained by using the secondary arylamine compound of the present invention is stable even when an oxidation state and a neutral state are repeated by an oxidation reaction and a reduction reaction subsequent to the oxidation. This means that the tertiary arylamine compound has resistance to repeated oxidation reactions. Accordingly, a light emitting element with high reliability can be obtained. By depositing the material for a light emitting element which is the tertiary arylamine compound obtained by using the secondary arylamine compound of the present invention, an amorphous film can be obtained. Therefore, a long-life light emitting element can be obtained.

### EMBODIMENT MODE 4

Embodiment Mode 4 will describe a light emitting device which is manufactured by using a material for a light emitting element of the present invention.

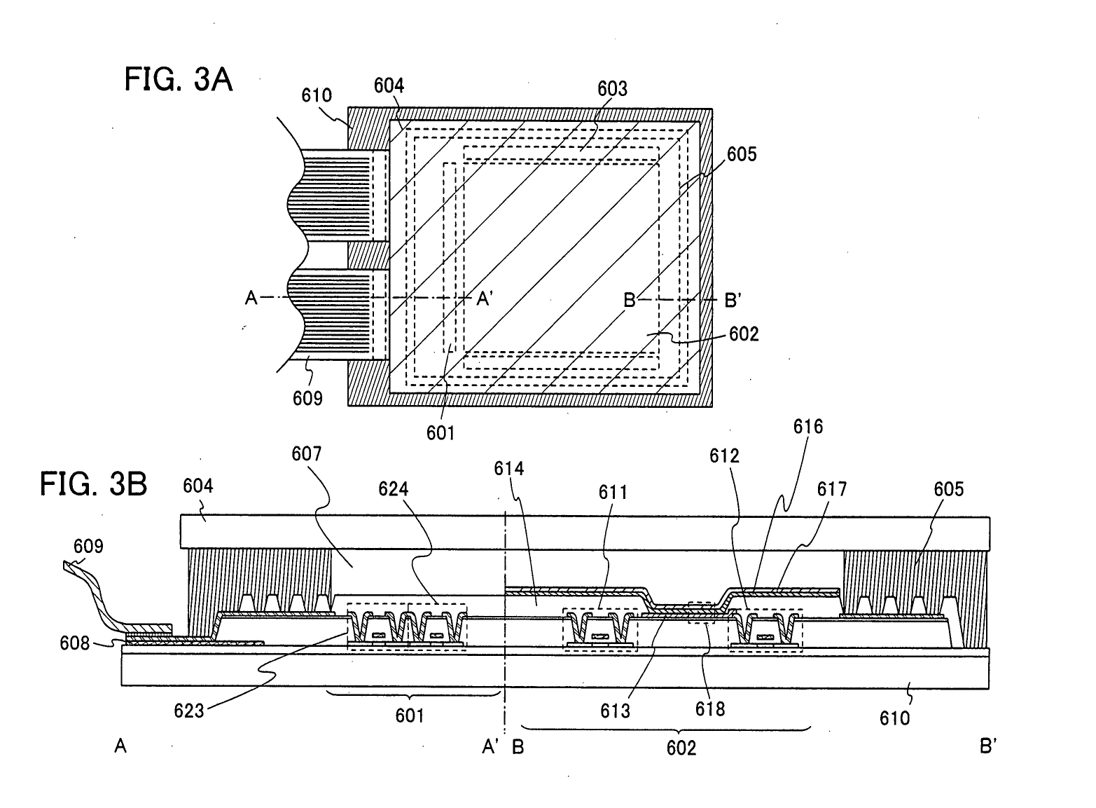

In Embodiment Mode 4, a light emitting device which is manufactured by using a material for a light emitting element of the present invention will be described with reference to FIGS. 3A and 3B. FIG. 3A is a top view of the light emitting device, and FIG. 3B is a cross sectional view taken along A-A′ and B-B′ of FIG. 3A. A portion 601 surrounded by a dotted line is a driver circuit portion (source side driver circuit), a portion 602 surrounded by another dotted line is a pixel portion, and a portion 603 surrounded by further another dotted line is a driver circuit portion (gate side driver circuit). In addition, reference numeral 604 denotes a sealing substrate, and 605: a sealant, and the inside surrounded by the sealant 605 is a space 607.

A leading wire 608 is a wire for transmitting signals to be input to the source side driver circuit 601 and the gate side driver circuit 603, and receives a video signal, a clock signal, a start signal, a reset signal, or the like from an FPC (flexible printed circuit) 609 as an external input terminal. Although only the FPC is shown here, a printed wiring board (PWB) may be attached to the FPC. The light emitting device in this specification includes not only the light emitting device itself but also a state in which the FPC or the PWB is attached thereto.

Next, a sectional structure will be described with reference to FIG. 3B. The driver circuit portions and the pixel portion are formed on an element substrate 610, however, the source side driver circuit 601 which is the driver circuit portion and one pixel of the pixel portion 602 are shown here.

In the source side driver circuit 601, a CMOS circuit in which an n-channel TFT 623 and a p-channel TFT 624 are combined is formed. The TFT forming the driver circuit may be formed of various kinds of CMOS circuit, PMOS circuit, or NMOS circuit. It is not always necessary to form the driver circuit on the substrate integrally as this embodiment mode, and it is also possible to form the driver circuit not on the substrate but outside the substrate externally.

The pixel portion 602 includes a plurality of pixels, each of which includes a switching TFT 611, a current control TFT 612, and a first electrode 613 electrically connected to a drain of the current control TFT 612. An insulator 614 is formed to cover an end portion of the first electrode 613. Here, a positive photosensitive acrylic resin film is used to form the insulator 614.

In addition, an upper or lower end portion of the insulator 614 is made to have a curved surface with a curvature in order to improve the coverage. For example, in the case of using positive photosensitive acrylic as a material of the insulator 614, it is preferable that only the upper end portion of the insulator 614 be made to have a curved surface with a curvature radius (of 0.2 μm to 3 μm). Besides, as the insulator 614, it is possible to use either of a negative type resin which is insoluble in an etchant due to light and a positive type resin which is soluble in an etchant due to light.

On the first electrode 613, a layer containing a light emitting substance 616 and a second electrode 617 are formed. Here, it is preferable to use a material having a large work function as a material to be used for the first electrode 613 which functions as an anode. For example, it is possible to use a stacked layer of a titanium nitride film and a film containing aluminum as its main component, a three-layered structure of a titanium nitride film, a film containing aluminum as its main component, and a titanium nitride film, or the like, as well as a single-layer film such as an ITO film, an indium tin oxide film containing silicon, an indium oxide film containing zinc oxide of 2 to 20 wt %, a titanium nitride film, a chromium film, a tungsten film, a Zn film, or a Pt film. When the first electrode 613 has a stacked structure, it can have low resistance as a wire, form a favorable ohmic contact, and function as an anode.

The layer containing a light emitting substance 616 is formed by various kinds of methods such as an evaporation method with an evaporation mask, an inkjet method, and a spin coating method. The layer containing the light emitting substance 616 contains the material for a light emitting element of the present invention described in Embodiment Mode 2. As another material for forming the layer containing the light emitting substance 616, a low molecular material, an intermediate molecular material (including an oligomer and an dendrimer), or a high molecular material may be used. In addition, as a material used for the layer containing the light emitting substance, normally, an organic compound is often used as a single layer or a stacked layer. However, the present invention includes a structure in which an inorganic compound is used for a part of a film containing an organic compound.

As a material used for the second electrode 617 which serves as a cathode and is formed over the layer containing the light emitting substance 616, it is preferable to use a material having a small work function (Al, Mg, Li, Ca, an alloy or a compound thereof such as MgAg, MgIn, AlLi, LiF, or CaF₂). When light generated in the layer containing the light emitting substance 616 is made to pass through the second electrode 617, a stacked layer of a metal thin film with a thinned thickness and a transparent conductive film (ITO, indium oxide containing zinc oxide of 2 to 20 wt %, indium tin oxide containing silicon, zinc oxide (ZnO), or the like) may be used as the second electrode 617.

The sealing substrate 604 and the element substrate 610 are bonded with the sealant 605 to have a structure where a light emitting element 618 is provided in the space 607 which is surrounded by an element substrate 610, the sealing substrate 604, and the sealant 605. The space 607 is filled with a filler. There is a case in that the space 607 is filled with the sealant 605 in addition to the case in that the space 607 is filled with an inert gas (nitrogen, argon, or the like).

It is preferable to use an epoxy resin as the sealant 605. A material which does not transmit moisture and oxygen as much as possible is desirable. Further, as a material used for the sealing substrate 604, a plastic substrate containing FRP (fiberglass-reinforced plastics), PVF (polyvinyl fluoride), mylar, polyester, acrylic, or the like can be used, in addition to a glass substrate and a quartz substrate.

In the above-described manner, the light emitting device manufactured by using the material for a light emitting element of the present invention can be obtained.

Since the light emitting device of the present invention uses the material for a light emitting element described in Embodiment Mode 2, a light emitting device with favorable characteristics can be obtained. Specifically, a light emitting device whose power consumption is reduced can be obtained. In addition, a long-life light emitting device with high reliability can be obtained.

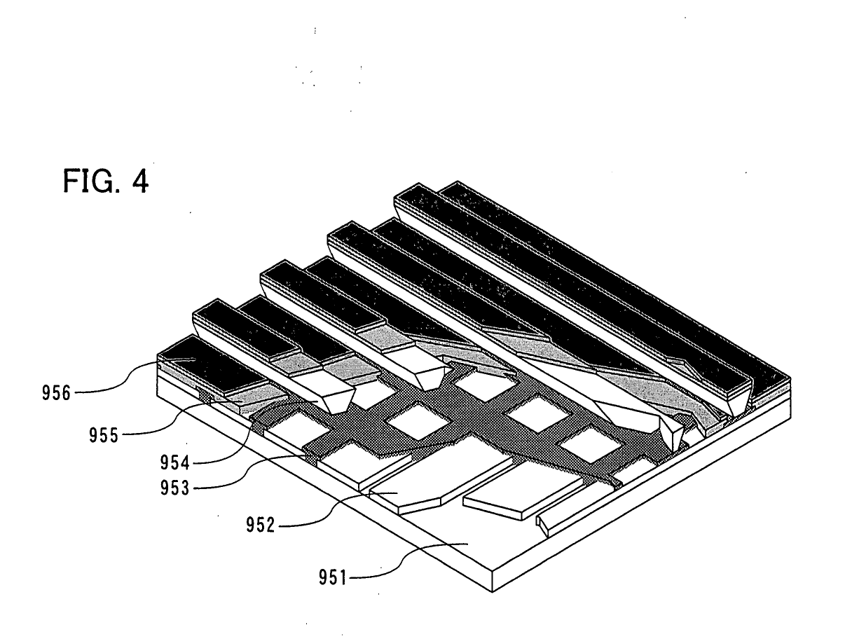

In this embodiment mode, an active light emitting device which controls driving of a light emitting element by using a transistor is described. Alternatively, a passive light emitting device which drives a light emitting element without being provided with an element for driving such as a transistor may be used. FIG. 4 is a perspective view of a passive light emitting device manufactured in accordance with the present invention. In FIG. 4, a layer containing a light emitting substance 955 is provided between an electrode 952 and an electrode 956, over a substrate 951. An end portion of the electrode 952 is covered by an insulating layer 953. Over the insulating layer 953, a partition wall layer 954 is provided. The partition wall layer 954 has a slope shape such that the distance between one sidewall and other side of the partition wall layer 954 is reduced toward a surface of the substrate. In other words, a cross section of the partition wall layer 954 in a narrow side direction shows a trapezoid shape having a shorter base (a side parallel to a surface of the insulating layer 953 and contacting with the insulating layer 953) than an upper side (a side parallel to a surface of the insulating layer 953 and not contacting with the insulating layer 953). By providing the partition wall layer 954 in this manner, a defect of a light emitting element due to static electricity or the like can be prevented. A passive light emitting device can be also driven with small power consumption by including a light emitting element of the present invention which operates with low driving voltage.

### EMBODIMENT MODE 5

Embodiment Mode 5 describes an electronic device of the present invention which partially includes the light emitting device shown in Embodiment Mode 4. The electronic device of the present invention contains the material for a light emitting element shown in Embodiment Mode 2 and includes a display portion with low power consumption, high reliability, and long life.

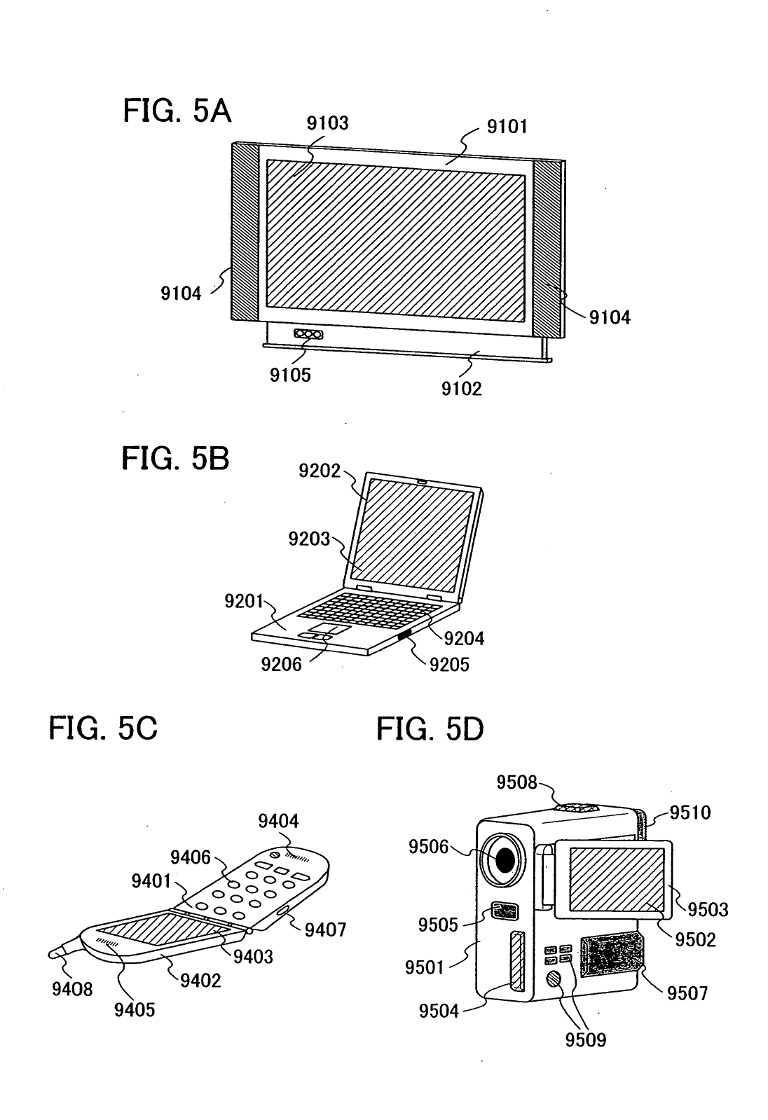

As examples of the electronic device including a light emitting element manufactured by using a material for a light emitting element of the present invention, there are a camera such as a video camera and a digital camera, a goggle display, a navigation system, an audio reproducing device (e.g., a car audio or audio component set), a computer, a game machine, a portable information terminal (e.g., a mobile computer, a mobile phone, a portable game machine, or an electronic book), an image reproducing device provided with a recording medium (specifically, a device for reproducing a recording medium such as a digital versatile disc (DVD) and having a display device for displaying the reproduced image), and the like. Such electronic devices are shown in FIGS. 5A to 5D.

FIG 5A shows a television set of the present invention, which includes a housing 9101, a supporting base 9102, a display portion 9103, speaker portions 9104, a video input terminal 9105, and the like. In this television set, the display portion 9103 has light emitting elements which are arranged in matrix and similar to the ones described in Embodiment Mode 3. The light emitting elements have characteristics of having low driving voltage, high reliability, and long life. Since the display portion 9103 formed of such light emitting elements has similar characteristics, this television set has less degradation in image quality and consumes low power. With such characteristics, the television set can have a significantly reduced number or size of a degradation correction function and power supply circuits. Therefore, the housing 9101 and the supporting base 9102 can be reduced in size and weight. Since the television set of the invention can achieve low power consumption, high image quality and reduction in size and weight, products suitable for any residential environment can be provided.

FIG. 5B shows a computer of the present invention, which includes a main body 9201, a housing 9202, a display portion 9203, a keyboard 9204, an external connecting port 9205, a pointing mouse 9206, and the like. In this computer, the display portion 9203 has light emitting elements which are arranged in matrix and similar to the ones described in Embodiment Mode 3. The light emitting elements have characteristics of having low driving voltage, high reliability, and long life. Since the display portion 9203 formed of such light emitting elements has similar characteristics, this computer has less degradation in image quality and consumes low power. With such characteristics, the computer can have a significantly reduced number or size of a degradation correction function and power supply circuits. Therefore, the main body 9201 and the housing 9202 can be reduced in size and weight. Since the computer of the invention can achieve low power consumption, high image quality and reduction in size and weight, products suitable for any environment can be provided.

FIG. 5C shows a portable phone of the present invention, which includes a main body 9401, a housing 9402, a display portion 9403, an audio input portion 9404, an audio output portion 9405, an operation key 9406, an external connecting port 9407, an antenna 9408, and the like. In this portable phone, the display portion 9403 has light emitting elements which are arranged in matrix and similar to the ones described in Embodiment Mode 3. The light emitting elements have characteristics of having low driving voltage, high reliability, and long life. Since the display portion 9403 formed of such light emitting elements has similar characteristics, this portable phone has less degradation in image quality and consumes low power. With such characteristics, the portable phone can have a significantly reduced number or size of a degradation correction function and power supply circuits. Therefore, the main body 9401 and the housing 9402 can be reduced in size and weight. Since the portable phone of the invention can achieve low power consumption, high image quality and reduction in size and weight, products suitable for carrying can be provided.

FIG. 5D shows a camera of the present invention, which includes a main body 9501, a display portion 9502, a housing 9503, an external connecting port 9504, a remote controller receiving portion 9505, an image receiving portion 9506, a battery 9507, an audio input portion 9508, operation keys 9509, an eye piece portion 9510, and the like. In this camera, the display portion 9502 has light emitting elements which are arranged in matrix and similar to the ones described in Embodiment Mode 3. The light emitting elements have characteristics of having low driving voltage, high reliability, and long life. Since the display portion 9502 formed of such light emitting elements has similar characteristics, this camera has less degradation in image quality and consumes low power. With such characteristics, the camera can have a significantly reduced number or size of a degradation correction function and power supply circuits. Therefore, the main body 9501 can be reduced in size and weight. Since the camera of the invention can achieve low power consumption, high image quality and reduction in size and weight, products suitable for carrying can be provided.

As described above, the applicable range of the light emitting device of the invention is so wide that the light emitting device can be applied to electronic devices in various fields. By using the material for a light emitting element of the present invention, electronic devices having display portions with low power consumption, high reliability and long life can be provided.

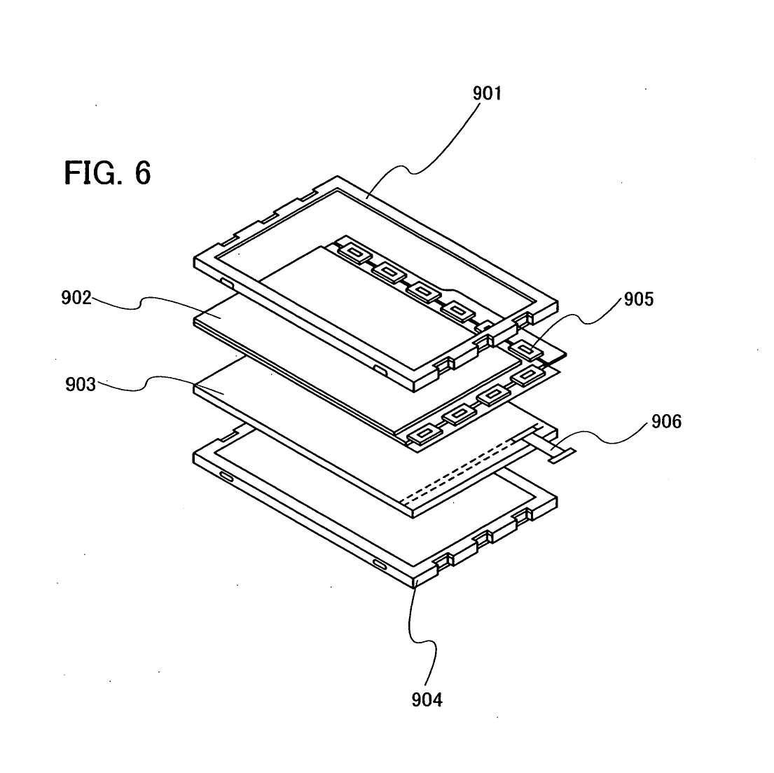

The light emitting device of the present invention can also be used as a lighting installation. One mode using the light emitting element of the present invention as a lighting installation will be described with reference to FIG. 6.

FIG. 6 shows an example of a liquid crystal display device using the light emitting device of the present invention as a backlight. The liquid crystal display device shown in FIG. 6 includes a housing 901, a liquid crystal layer 902, a backlight 903 and a housing 904, and the liquid crystal layer 902 is connected to a driver IC 905. The light emitting device of the present invention is used for the backlight 903, and current is supplied by a terminal 906.

By using the light emitting device of the present invention as the backlight of the liquid crystal display device, a backlight with reduced power consumption can be obtained. The light emitting device of the present invention is a plane emission type lighting installation, and can have a large area. Therefore, the backlight can have large area, and a liquid crystal display device having a large area can be obtained. Furthermore, since the light emitting device has a thin shape and consumes low power, a thin shape and low power consumption of a display device can also be achieved.

### EXAMPLE 1

Example 1 will describe a secondary arylamine compound of the present invention and a synthetic method thereof.

[Step 1]

First, a synthetic method of N-(4-diphenylaminophenyl)-N-(1-naphthyl)amine is described. A synthetic scheme of N-(4-diphenylaminophenyl)-N-(1-naphthyl)amine is shown in (A-2).

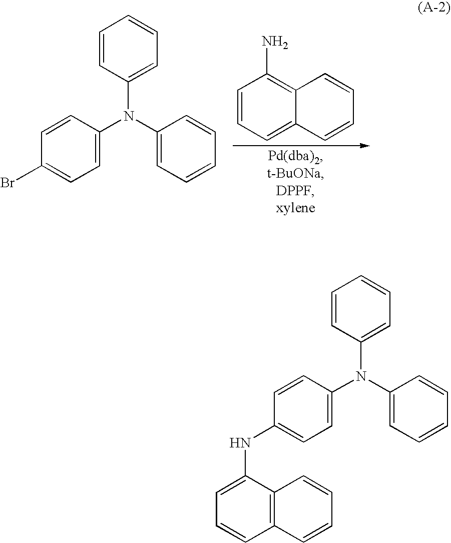

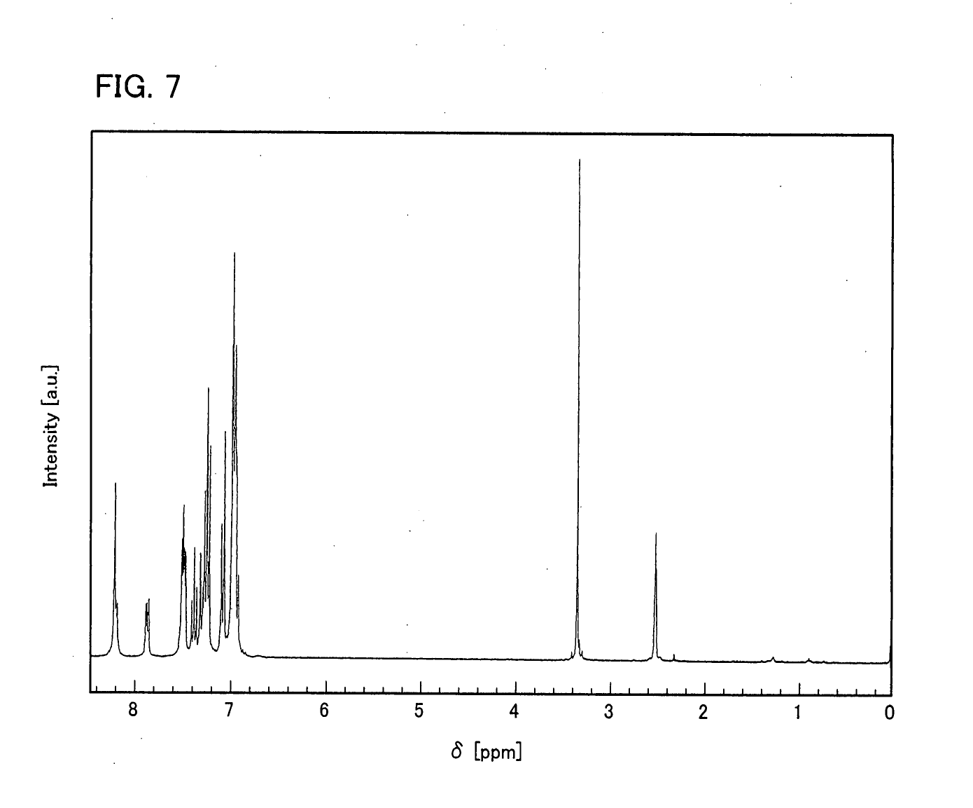

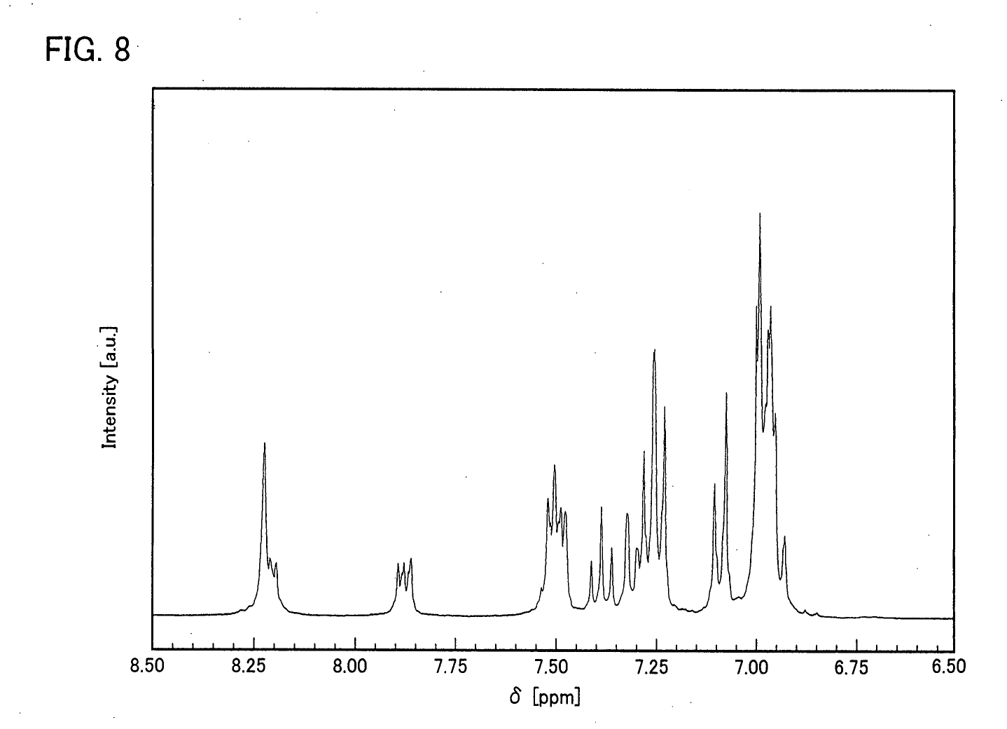

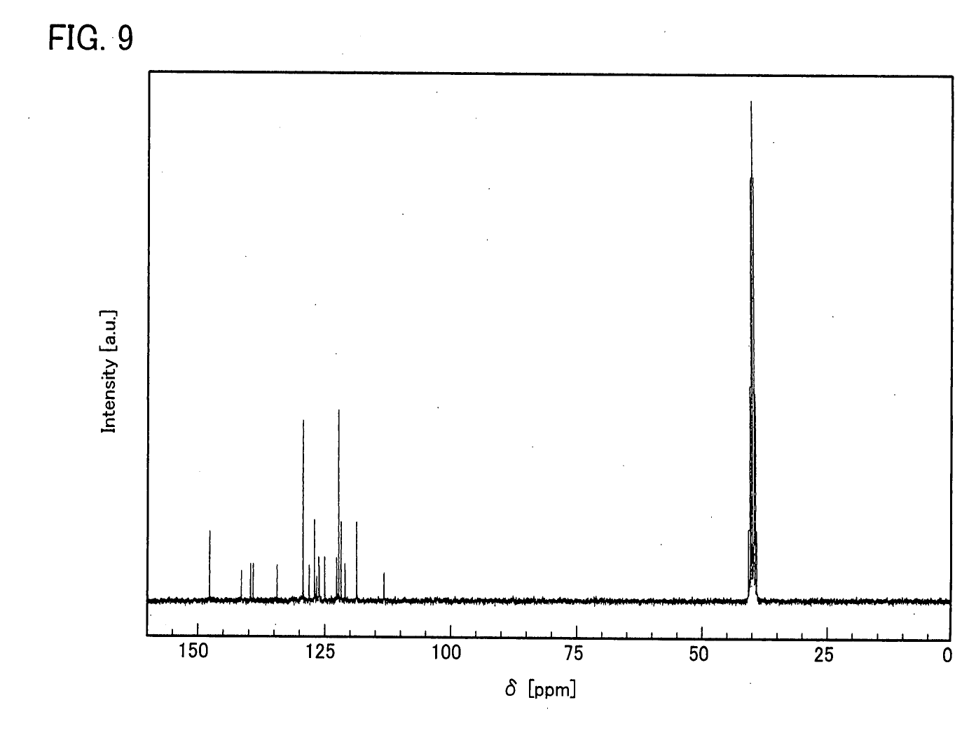

4-bromotriphenylamine (3.2 g, 10 mmol), 1-aminonaphthalene (1.4 g, 10 mmol), bis(dibenzylidene acetone)palladium(0) (58 mg, 0.1 mmol), and sodium tert-butoxide (3.0 g, 30 mmol) were mixed in a flask. After nitrogen was substituted for air in the flask, 40 ml of dehydrated xylene was added to the mixture in the flask. The mixture with the dehydrated xylene added was degassed for about 3 minutes until no bubbles were not generated from the mixture. Then, 1,1-bis(diphenylphosphino)ferrocene (540 mg, 1.0 mmol) was added to the mixture and the mixture was stirred, while being heated at 90° C., for 6.5 hours under a nitrogen atmosphere. Then, about 300 mL of toluene was added to the mixture, and the mixture was filtered through florisil, alumina and celite. The filtrate thus obtained was washed with water and saturated saline. Magnesium sulfate was added to the obtained organic phase, and the organic phase was then dried. The product was filtered and the filtrate was condensed. The condensed filtrate was purified by using silica gel column chromatography (toluene:hexane=3:7). The fraction thus obtained was condensed and hexane was added. Ultrasonication was applied to the product, to generate a solid. The obtained solid was filtered out, and 1.8 g of a white powder of N-(4-diphenylaminophenyl)-N-(1-naphthyl)amine was obtained in a yield of 46%. The NMR data is as follows: ¹H NMR (300 MHz, DMSO-d); δ=6.93-7.00 (m, 8H), 7.09 (d, j=8.7, 2 H), 7.23-7.32 (m, 5 H), 7.39 (t, j=7.8, 1 H), 7.48-7.52 (m, 3H), 7.86-7.90 (m, 1 H), 8.20-8.23 (m, 2H). ¹³C NMR (60 MHz, DMSO-d); δ=113.2, 118.6, 120.9, 121.7, 122.2, 122.6, 125.0, 126.0, 126.2, 126.6, 127.0, 128.1, 129.3, 134.4, 139.1, 139.6, 141.4, 147.6. Further, a chart of ¹H-NMR is shown in FIG. 7. In addition, FIG. 8 is a chart showing an enlarged version of the 6.5 to 8.5 ppm range section of FIG. 7. A chart of ¹³C-NMR is shown in FIG. 9.

### EXAMPLE 2

As an example of a derivative which uses a secondary arylamine compound of the present invention, 3-[N-(4-diphenylaminophenyl)-N-(1-naphthyl)amino]-9-phenylcarbazole (PCzTPN1), which is represented by Structural Formula 61, and a synthetic method thereof are described. 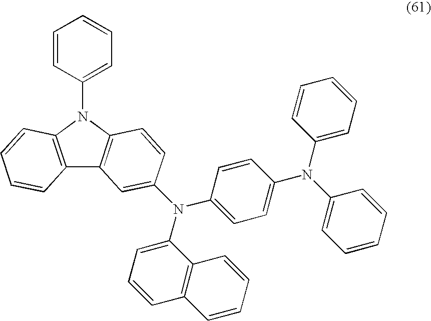
[Step 1]

A synthetic scheme of 3-[N-(4-diphenylaminophenyl)-N-(1-naphthyl)amino]-9-phenylcarbazole (PCzTPN1) is shown in (B-1).

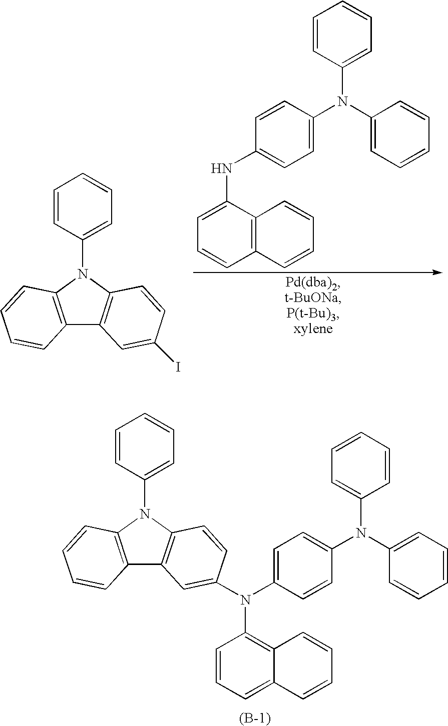

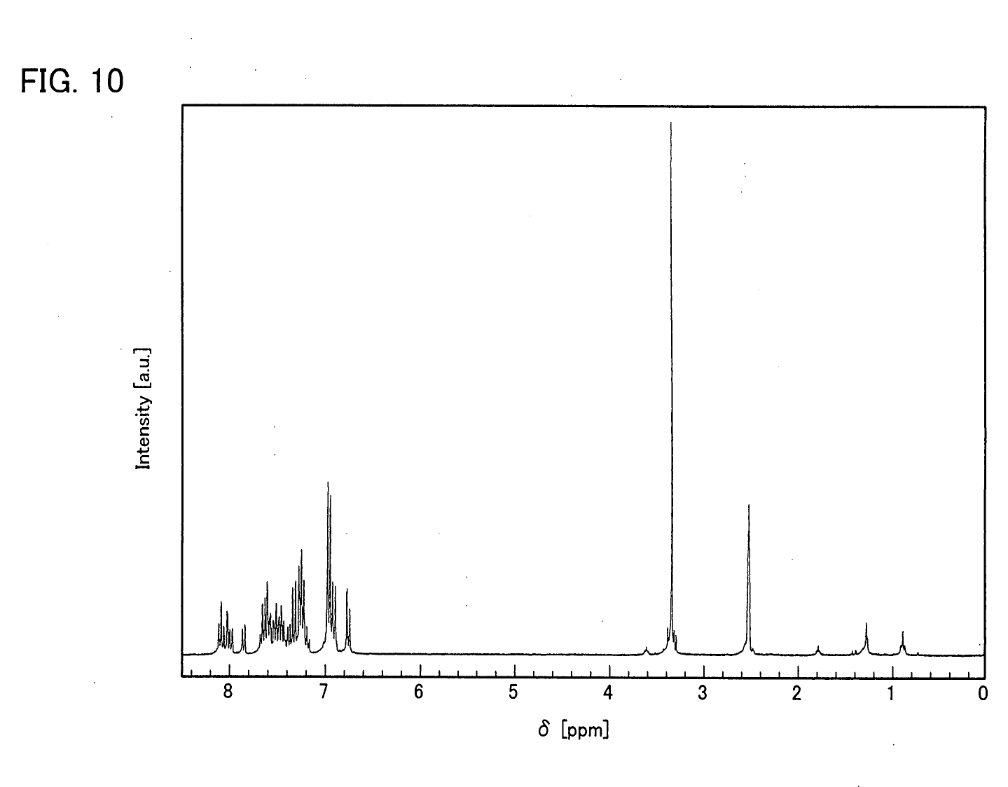

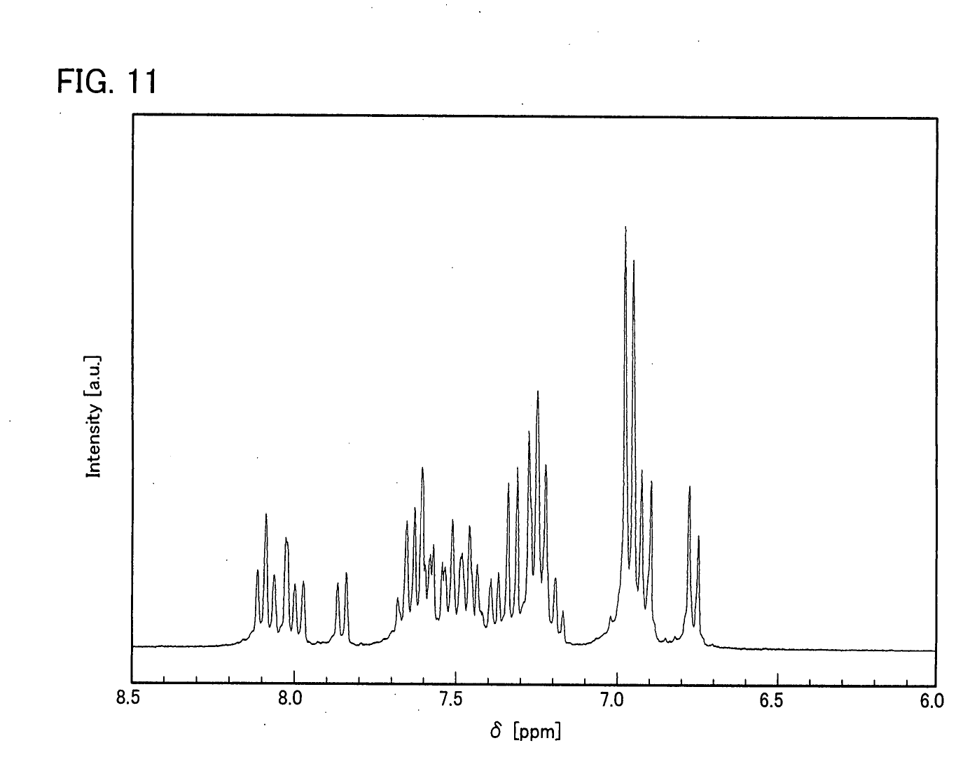

740 mg (2.0 mmol) of 3-iodine-9-phenylcarbazole, 700 mg (1.8 mmol) of N-(4-diphenylaminophenyl)-N-(1-naphthyl)amine, 12 mg (0.02 mmol) of bis(dibenzylidene acetone)palladium(0), and 600 mg (6.0 mmol) of sodium tert-butoxide were mixed in a flask. Nitrogen was substituted for air in the flask. Then, 5 mL of dehydrated xylene was added to the mixture, and the mixture was degassed for about 3 minutes. Next, 0.1 mL (0.05 mmol) of tri-tert-butyl phosphine (a 10 wt % hexane solution) was added to the mixture and the content was stirred for 5.5 hours, while being heated at 90° C., under a nitrogen atmosphere. Then, about 100 mL of toluene was added to the mixture, and the mixture was filtered through florisil, alumina and celite. The filtrate thus obtained was washed with water and saturated saline. Magnesium sulfate was added to the organic phase thus obtained, and the organic phase was then dried. The product was filtered and filtrate was condensed. The condensed filtrate was purified by using a silica gel column chromatography (toluene:hexane=3:7). The thus obtained fraction was condensed and added with hexane. The product was applied with supersonication. Subsequently, the product was filtered to obtain a solid that is 500 mg of a cream-colored powder of PCzTPN1. The yield was 44%. The NMR data is as follows: ¹H NMR (300 MHz, DMSO-d); δ=6.74 (d, j=8.7, 2H), 6.88-7.00 (m, 8H), 7.16-7.67 (m, 23H), 7.84 (d, j=8.4, 1H), 7.97 (d, j=8.1, 1H), 8.02 (s, 1H), 8.08 (t, j=7.8, 2H). Also, a chart of ¹H-NMR is shown in FIG. 10. In addition, FIG. 11 is a chart showing an enlarged version of the 6.0 to 8.5 ppm range section of FIG. 10.

Thermogravimetry-differential thermal analysis (TG-DTA) of the obtained PCzTPN1 was carried out. A thermo-gravimetric/differential thermal analyzer (manufactured by Seiko Instruments Inc., TG/DTA-320) was used for the measurement, and a thermophysical property of the PCzTPN1 was evaluated at a rate of temperature increase of 10° C./min under a nitrogen atmosphere. As a result, the temperature at which the weight decreased to 95% or less of the weight at the beginning of the measurement under normal pressure was found to be 380° C., according to a relation between weight and temperature (thermogravimetric analysis).

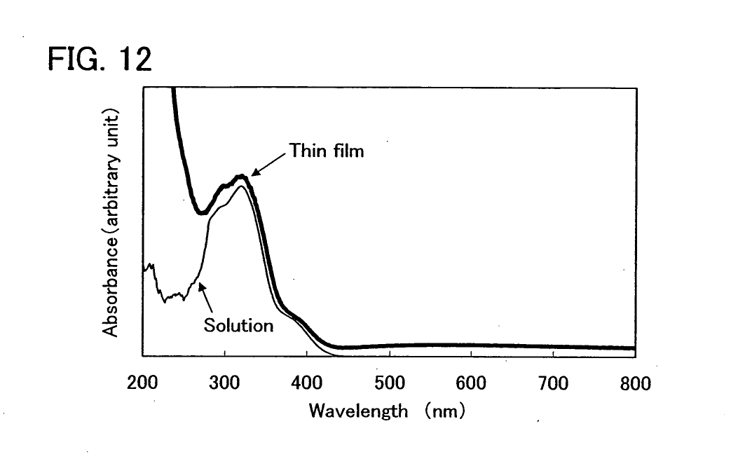

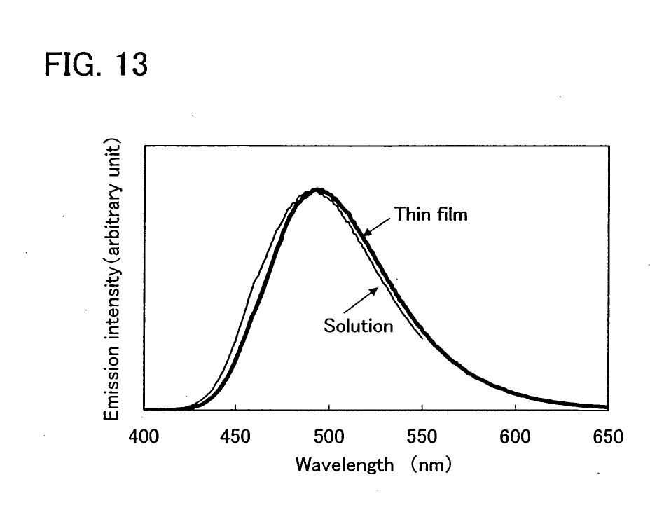

Absorption spectra of the toluene solution of PCzTPN1 and of a thin film of PCzTPN1 are shown in FIG. 12. A UV/VIS spectrophotometer (manufactured by JASCO Corporation, V-550) was used for the measurement. In FIG. 12, the horizontal axis indicates wavelength (nm) and the vertical axis indicates absorbance (arbitrary measurement unit). The maximum absorption wavelength was 314 nm in the case of the toluene solution, and 314 nm in the case of the thin film. Emission spectra of the toluene solution (excitation wavelength: 330 nm) of PCzTPN1 and the thin film (excitation wavelength: 350 nm) of PCzTPN1 are shown in FIG. 13. In FIG. 13, the horizontal axis indicates wavelength (nm) and the vertical axis indicates emission intensity (arbitrary measurement unit). The maximum emission wavelength was 490 nm (excitation wavelength: 330 nm) in the case of the toluene solution, and 500 nm (excitation wavelength: 350 nm) in the case of the thin film.

Further, the HOMO level and the LUMO level of PCzTPN1 in a state of a thin film were measured. A value of the HOMO level was obtained by converting a value of ionization potential measured by a photoelectron spectrometer (manufactured by Riken Keiki Co., Ltd., AC-2) into a negative value. A value of the LUMO level was obtained by using an absorption edge of the thin film in FIG. 12 as an energy gap and adding the value of the absorption edge to the value of the HOMO level. As a result, the HOMO level and the LUMO level were found to be −5.21 eV and −2.28 eV, respectively.

An oxidation reaction property of PCzTPN1 was measured by cyclic voltammetry (CV) measurement, using an electrochemical analyzer (manufactured by BAS Inc., ALS model 600A).

The solution for the CV measurement was prepared by using dehydrated dimethylformamide (DMF) (manufactured by Aldrich Chemical Company, 99.8%, catalog number: 22705-6) as a solvent, dissolving a supporting electrolyte of tetra-n-butylammonium perchlorate (n-Bu₄NClO₄) (manufactured by Tokyo Chemical Industry Co., LTD., catalog number: T0836) to a concentration of 100 mmol/L, and dissolving the material to be measured to a concentration of 1 mmol/L. A platinum electrode (manufactured by BAS Inc., PTE platinum electrode) was used as a working electrode, another platinum electrode (manufactured by BAS Inc., Pt counter electrode for VC-3, (5 cm)) was used as an auxiliary electrode, and an Ag/Ag⁺ electrode (manufactured by BAS Inc., RE-5 reference electrode for nonaqueous solvent) was used as a reference electrode. The measurement was carried out at room temperature.

The oxidation reaction property of PCzTPN1 was measured as follows. The scan in which the potential of the working electrode with respect to the reference electrode was changed from −0.03 to 0.4 V, and then changed from 0.4 to −0.03 V, is referred to as one cycle. The oxidation reaction property of PCzTPN1 for 100 cycles was measured. The CV measurement was carried out with a scan speed of 0.1 V/s.

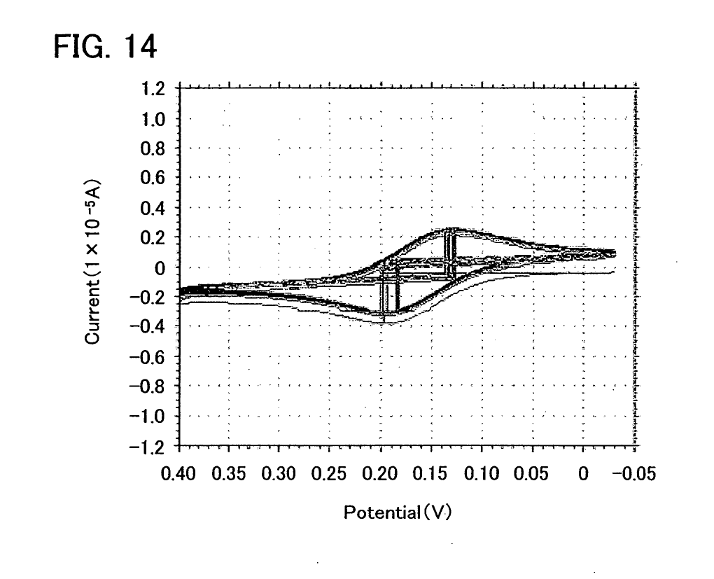

FIG. 14 shows the result of the measurement of the oxidation reaction property of PCzTPN1. In FIG. 14, the horizontal axis shows the potential (V) of the working electrode with respect to the reference electrode, and the vertical axis shows a value of a current (1×10⁻⁵ A) flowing between the working electrode and the auxiliary electrode.

From FIG. 14, it was found that the oxidation potential was 0.20 V (vs. Ag/Ag⁺ electrode). After 100 cycles of scanning were carried out, the peak position and the peak intensity of the CV curve hardly changed. Therefore, it can be said that the material for a light emitting element according to the present invention is quite stable in an oxidation reaction.

### EXAMPLE 3

As an example of a derivative which uses a secondary arylamine compound of the present invention, 3,6-bis[N-(4-diphenylaminophenyl)-N-(1-naphthyl)amino]-9-phenylcarbazole (PCzTPN2) which is represented by Structural Formula 62, and a synthetic method thereof are described. 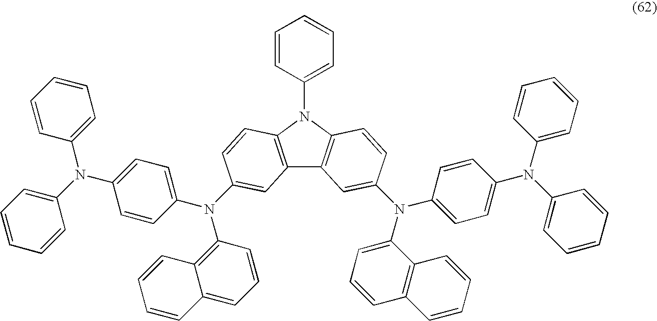
[Step 1]

A synthetic scheme of 3,6-bis[N-(4diphenylaminophenyl)-N-(1-naphthyl)amino]-9-phenylcarbazole (PCzTPN2) is shown in (B-2).

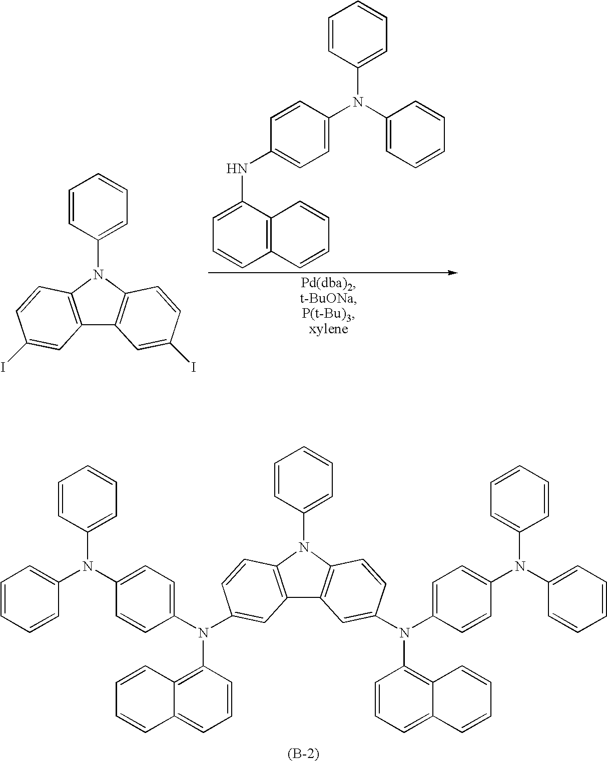

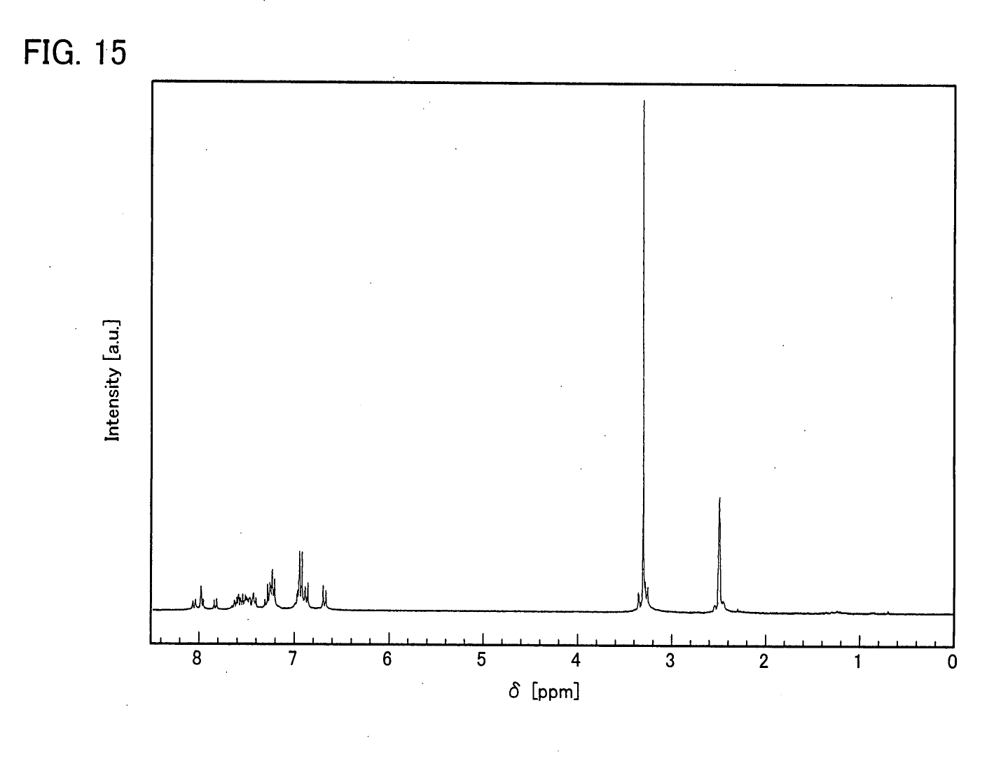

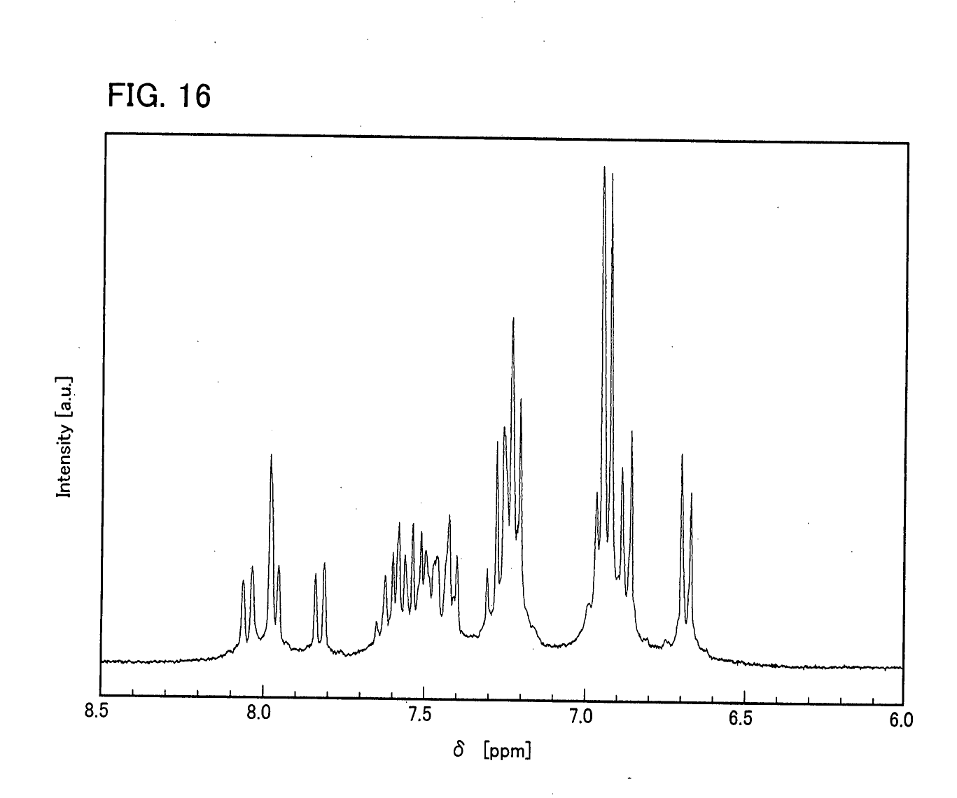

740 mg (1.5 mmol) of 3,6-diiodo-9-phenylcarbazole, 1.2 g (3 mmol) of N-(4-diphenylaminophenyl)-N-(1-naphthyl)amine, 18 mg (0.03 mmol) of bis(dibenzylidene acetone)palladium(0), and 1.0 g (10 mmol) of sodium tert-butoxide were mixed in a flask. After nitrogen was substituted for air in the flask, 7.5 ml of dehydrated xylene was added to the mixture and the mixture was degassed for 3 minutes. Then, 0.2 ml (0.1 mmol) of tri-tert-butyl phosphine (a 10 wt % hexane solution) was added to the mixture. The mixture was stirred, while being heated at 90° C., for 7 hours under a nitrogen atmosphere. Further, about 300 mL of toluene was added to the mixture, and then the mixture was filtered through florisil, alumina and celite. The filtrate thus obtained was washed with water and saturated saline. Magnesium sulfate was added to the organic phase thus obtained, and the organic phase was then dried. The product was filtered and the filtrate was condensed. The condensed filtrate was purified by using silica gel column chromatography (toluene:hexane=3:7). The fraction thus obtained was condensed and hexane was added. Ultrasonic was applied to the product, to generate a solid. The obtained solid was filtered out, and 1.0 mg of a yellow powder of PCzTPN2 was obtained. The yield was 66%. The NMR data is as follows: ¹H NMR (300 MHz, DMSO-d) ; δ=6.68 (d, j=9.0, 4H), 6.86-6.97 (m, 16H), 7.20-6.97 (m, 16H), 7.20-7.65 (m, 25H), 7.83 (d, j=8.1, 2H), 7.95-7.98 (m, 4H), 8.05 (d, j=8.4, 2H). Further, a chart of ¹H-NMR is shown in FIG. 15. In addition, FIG. 16 is a chart showing an enlarged version of the 6.0 to 8.5 ppm range section of FIG. 15.

Thermogravimetry-differential thermal analysis (TG-DTA) of the obtained PCzTPN2 was carried out. A thermo-gravimetric/differential thermal analyzer (manufactured by Seiko Instruments Inc., TG/DTA-320) was used for the measurement, and a thermophysical property of the obtained PCzTPN2 was evaluated at a rate of temperature increase of 10° C./min under a nitrogen atmosphere. As a result, the temperature at which the weight decreased to be 95% or less of the weight at the beginning of the measurement under normal pressure was found to be 470° C., according to a relation between weight and temperature (thermogravimetric analysis).

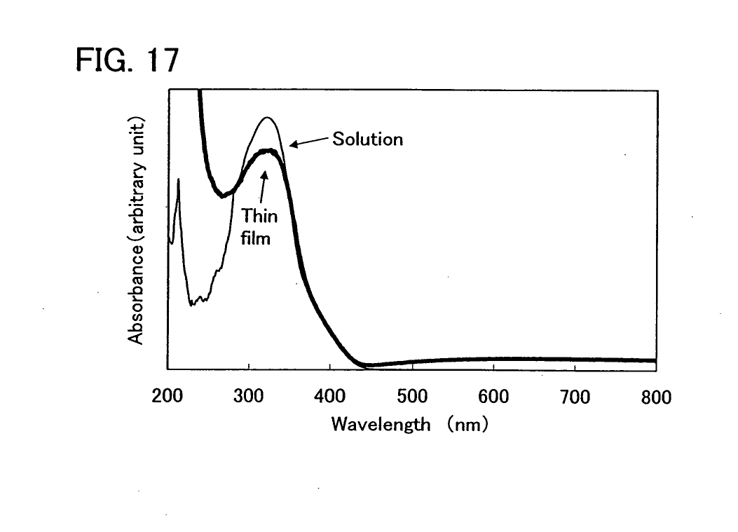

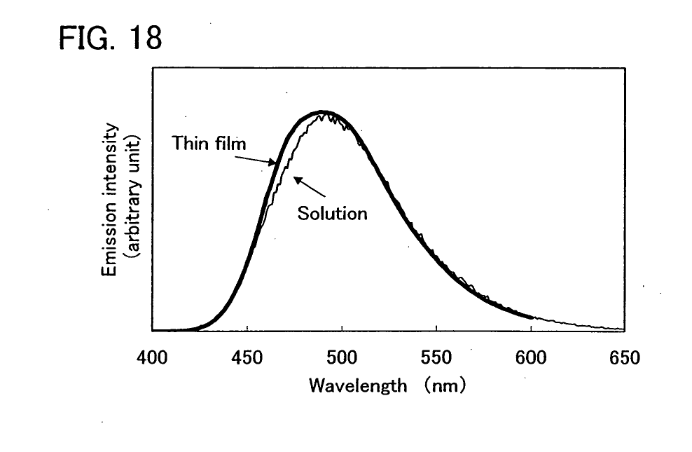

Absorption spectra of the toluene solution of PCzTPN2 and of a thin film of PCzTPN2 are shown in FIG. 17. An UV/VIS spectrophotometer (manufactured by JASCO Corporation, V-550) was used for the measurement. In FIG. 17, the horizontal axis indicates wavelength (nm) and the vertical axis indicates absorbance (arbitrary measurement unit). The maximum absorption wavelength was 320 nm in the case of the toluene solution, and 393 nm in the case of the thin film. Emission spectra of the toluene solution (excitation wavelength: 335 nm) of PCzTPN2 and of the thin film (excitation wavelength: 320 nm) of PCzTPN1 are shown in FIG. 18. In FIG. 18, the horizontal axis indicates wavelength (nm) and the vertical axis indicates emission intensity (arbitrary measurement unit). The maximum emission wavelength was 493 nm (excitation wavelength: 335 nm) in the case of the toluene solution, and 488 nm (excitation wavelength: 320 nm) in the case of the thin film.

Further, the HOMO level and the LUMO level of PCzTPN2 in a state of a thin film were measured. A value of the HOMO level was obtained by converting a value of ionization potential measured by a photoelectron spectrometer (manufactured by Riken Keiki Co., Ltd., AC-2) into a negative value. A value of the LUMO level was obtained by using an absorption edge of the thin film in FIG. 17 as an energy gap and adding the value of the absorption edge to the value of the HOMO level. As a result, the HOMO level and the LUMO level were found to be −5.13 eV and −2.24 eV, respectively.

An oxidation reaction property of PCzTPN2 was measured by cyclic voltammetry (CV) measurement, using an electrochemical analyzer (manufactured by BAS Inc., ALS model 600A).

The solution for the CV measurement was prepared by using dehydrated dimethylformamide (DMF) (manufactured by Aldrich Chemical Company, 99.8%, catalog number: 22705-6) as a solvent, dissolving a supporting electrolyte of tetra-n-butylammonium perchlorate (n-Bu₄NClO₄) (manufactured by Tokyo Chemical Industry Co., LTD., catalog number: T0836) to a concentration of 100 mmol/L, and dissolving the material to be measured to a concentration of 1 mmol/L. A platinum electrode (manufactured by BAS Inc., PTE platinum electrode) was used as a working electrode, another platinum electrode (manufactured by BAS Inc., Pt counter electrode for VC-3, (5 cm)) was used as an auxiliary electrode, and an Ag/Ag⁺ electrode (manufactured by BAS Inc., RE-5 reference electrode for nonaqueous solvent) was used as a reference electrode. The measurement was carried out at room temperature.

The oxidation reaction property of PCzTPN2 was measured as follows. A scan in which the potential of the working electrode with respect to the reference electrode was changed from −0.36 to 0.4 V, and then changed from 0.4 to −0.36 V, is referred to as one cycle. The oxidation reaction property of PCzTPN2 for 100 cycles was measured. The CV measurement was carried out with a scan speed of 0.1 V/s.

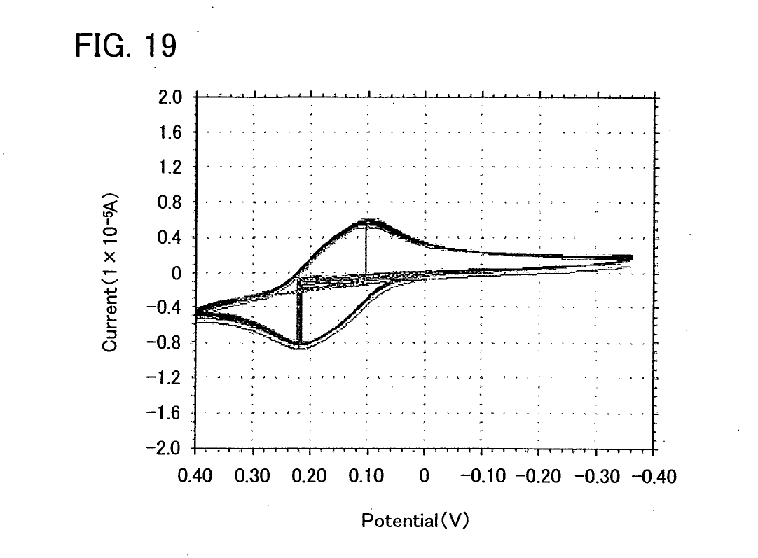

FIG. 19 shows the result of the measurement of the oxidation reaction property of PCzTPN2. In FIG. 19, the horizontal axis shows the potential (V) of the working electrode with respect to the reference electrode, and the vertical axis shows a value of a current (1×10⁻⁵ A) flowing between the working electrode and the auxiliary electrode.

From FIG. 19, it was found that the oxidation potential was 0.22 V (vs. Ag/Ag⁺ electrode). After 100 cycles of scanning were carried out, the peak position and the peak intensity of the CV curve hardly changed. Therefore, it can be said that the material for a light emitting element according to the present invention is quite stable in an oxidation reaction.

### EXAMPLE 4

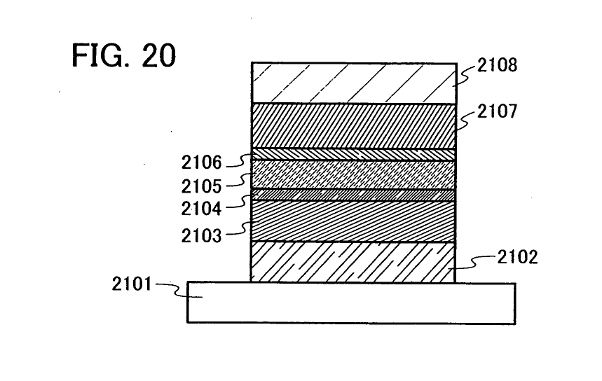

Example 4 will describe a light emitting element which is manufactured by using a material for a light emitting element of the present invention, with reference to FIG. 20.

First, a film of indium tin oxide containing silicon oxide was formed over a glass substrate 2101 by a sputtering method to form a first electrode 2102. Note that the thickness thereof was 110 nm and the electrode area was 2 mm×2 mm.

Next, the substrate provided with the first electrode was fixed to a substrate holder which was provided in a vacuum evaporation apparatus, so that a surface of the substrate on which the first electrode was formed faced downward. Then, the vacuum evaporation apparatus was evacuated so that the pressure was reduced to approximately 10⁻⁴ Pa, and PCzTPN1 and molybdenum oxide (VI) were co-evaporated over the first electrode 2102 to form a layer containing a composite material 2103. The thickness thereof was set to 50 nm, and a weight ratio of PCzTPN1 to molybdenum oxide (VI) was set at 4:2 (=PCzTPN1:molybdenum oxide). Note that a co-evaporation method is an evaporation method in which evaporation from a plurality of evaporation sources is performed concurrently in one treatment chamber.

Next, a film of NPB was formed to have a thickness of 10 nm by an evaporation method using resistance heating, to form a hole transporting layer 2104.

Further, by co-evaporating Alq and coumarin 6, a light emitting layer 2105 with a thickness of 40 nm was formed over the hole transporting layer 2104. Here, a weight ratio of Alq to coumarin 6 was set to be 1:0.01 (=Alq:coumarin 6).

After that, a film of Alq was formed with a thickness of 10 nm over the light emitting layer 2105 by an evaporation method using resistance heating, to form an electron transporting layer 2106.

Further, Alq and lithium were co-evaporated over the electron transporting layer 2106 so that an electron injecting layer 2107 was formed with a thickness of 30 nm over the Alq. Here, a weight ratio of Alq to lithium was set to be 1:0.01 (=Alq:lithium).

Finally, an aluminum film was formed with a thickness of 200 nm over the electron injecting layer 2107 by an evaporation method using resistance heating to form a second electrode 2108. Accordingly, the light emitting element of Example 4 was manufactured.

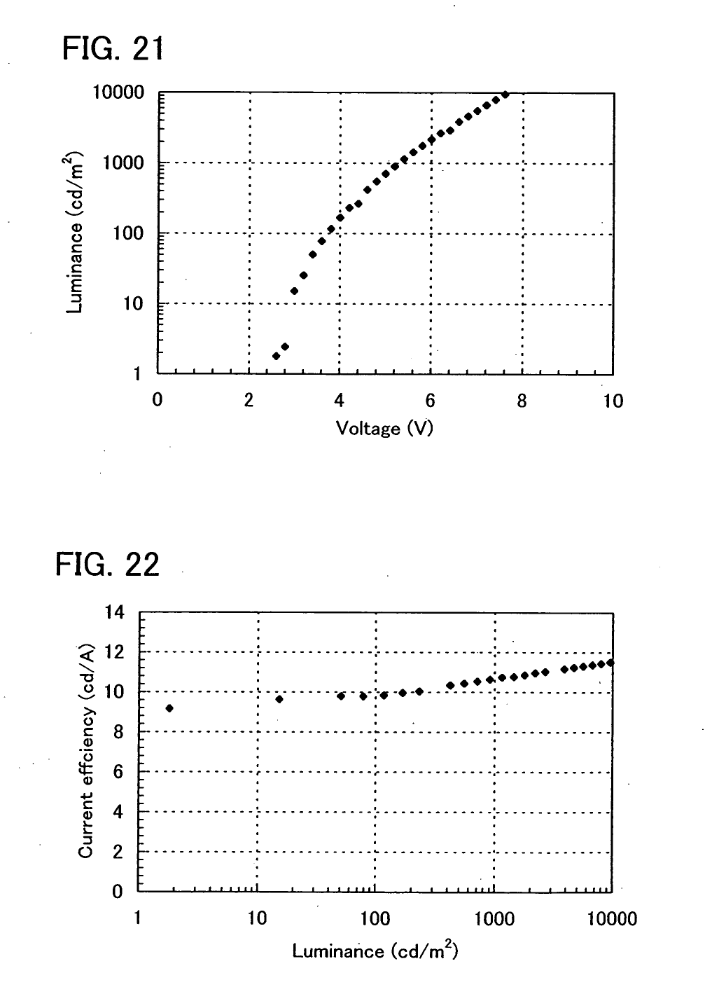

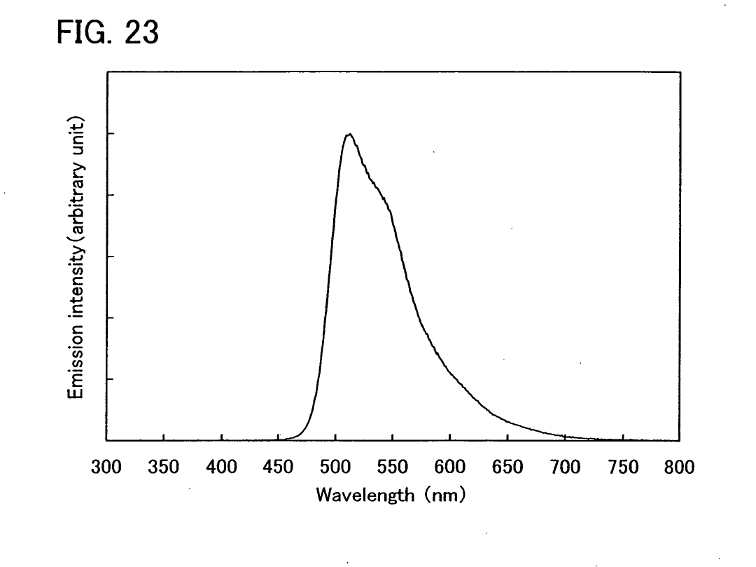

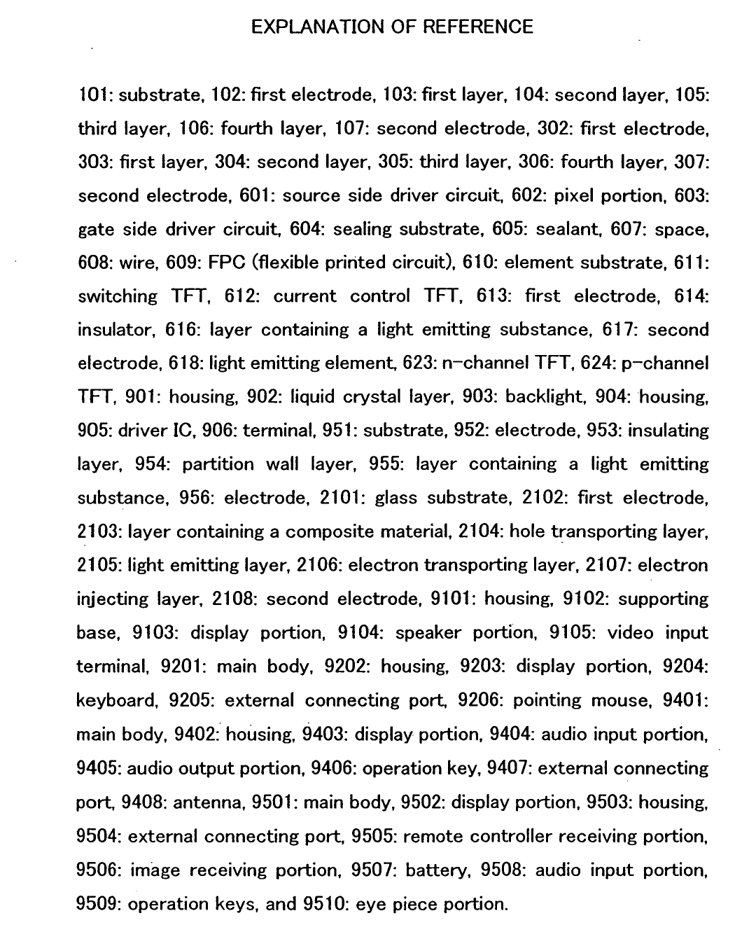

FIG. 21 shows luminance-voltage characteristics of the light emitting element of Example 4. In addition, FIG. 22 shows current efficiency-luminance characteristics of the light emitting element of Example 4. FIG. 23 shows an emission spectrum of the light emitting element of Example 4 when applied with a current of 1 mA. In the light emitting element of Example 4, the voltage required to obtain a luminance of 903 cd/m² was 5.2 V, the current flowing at the time was 0.34 mA (the current density was 8.5 mA/cm²), and the CIE chromaticity coordinates were (x=0.30, y=0.63). The current efficiency at that time was 10.6 cd/A.

As described above, since the material for a light emitting element of the present invention has an excellent hole transporting property, it can be used in a light emitting element, as a part of a composite material which also contains metal oxide. By using the composite material containing the material for a light emitting element of the present invention, ohmic contact with the first electrode can be realized, and the driving voltage of the light emitting element can be reduced.

This application is based on Japanese Patent Application serial no. 2005-234432 filed in Japan Patent Office on Aug. 12, 2005, the entire contents of which are hereby incorporated by reference.

1. A secondary arylamine compound represented by General Formula 1,
wherein Ar¹¹ is one of an aryl group having 7 to 25 carbon atoms and a heteroaryl group having 7 to 25 carbon atoms,
wherein each of Ar¹² and Ar¹³ is one of an aryl group having 6 to 25 carbon atoms and a heteroaryl group having 5 to 9 carbon atoms, and
wherein X is one of a bivalent aromatic hydrocarbon group having 6 to 25 carbon atoms and a bivalent heterocyclic group having 5 to 10 carbon atoms.

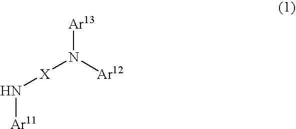

2. A secondary arylamine compound represented by General Formula 2,
wherein each of Ar²² and Ar²³ is one of an aryl group having 6 to 25 carbon atoms and a heteroaryl group having 5 to 9 carbon atoms, and
wherein X is one of a bivalent aromatic hydrocarbon group having 6 to 25 carbon atoms and a bivalent heterocyclic group having 5 to 10 carbon atoms.

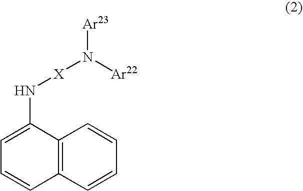

3. A secondary arylamine compound represented by Structural Formula 31.

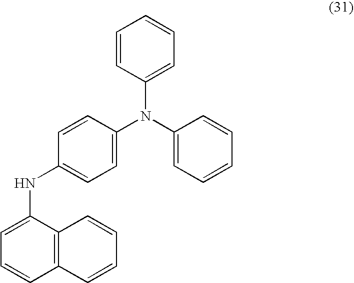

4. A material for a light emitting element, comprising, as a substituent, the secondary arylamine compound according to claim 1.

5. A material for a light emitting element, comprising, as a substituent, the secondary arylamine compound according to claim 2.

6. A material for a light emitting element, comprising, as a substituent, the secondary arylamine compound according to claim 3.

7. A material for a light emitting element, represented by General Formula 4,
wherein Ar¹¹ is one of an aryl group having 7 to 25 carbon atoms and a heteroaryl group having 7 to 25 carbon atoms,
wherein each of Ar¹² and Ar¹³ is one of an aryl group having 6 to 25 carbon atoms and a heteroaryl group having 5 to 9 carbon atoms,
wherein X is one of a bivalent aromatic hydrocarbon group having 6 to 25 carbon atoms and a bivalent heterocyclic group having 5 to 10 carbon atoms,
wherein R¹ is one selected from the group consisting of hydrogen, an alkyl group having 1 to 6 carbon atoms, an aryl group having 6 to 25 carbon atoms, a heteroaryl group having 5 to 9 carbon atoms, an arylalkyl group, and an acyl group having 1 to 7 carbon atoms, and
R² is one selected from the group consisting of hydrogen, an alkyl group having 1 to 6 carbon atoms, and a substituent represented by General Formula 5, wherein
Ar¹⁴ is one of an aryl group having 7 to 25 carbon atoms and a heteroaryl group having 7 to 25 carbon atoms,
each of Ar¹⁵ and Ar¹⁶ is one of an aryl group having 6 to 25 carbon atoms and a heteroaryl group having 5 to 9 carbon atoms, and
Y is one of a bivalent aromatic hydrocarbon group having 6 to 25 carbon atoms and a bivalent heterocyclic group having 5 to 10 carbon atoms.

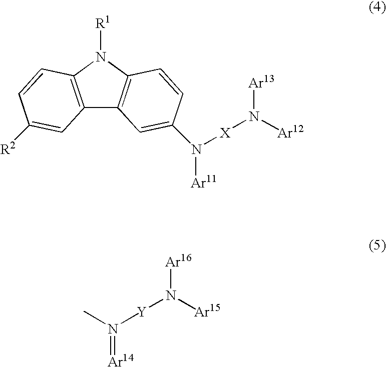

8. A material for a light emitting element represented by General Formula 6,
wherein each Ar²² and Ar²³ is one of an aryl group having 6 to 25 carbon atoms and a heteroaryl group having 5 to 9 carbon atoms,
wherein X is one of a bivalent aromatic hydrocarbon group having 6 to 25 carbon atoms and a bivalent heterocyclic group having 5 to 10 carbon atoms,
wherein R¹ is one selected from the group consisting of hydrogen, an alkyl group having 1 to 6 carbon atoms, an aryl group having 6 to 25 carbon atoms, a heteroaryl group having 5 to 9 carbon atoms, an arylalkyl group, and an acyl group having 1 to 7 carbon atoms, and
wherein R² is one selected from the group consisting of hydrogen, an alkyl group having 1 to 6 carbon atoms, and a substituent represented by General Formula 7, wherein
each of Ar²⁵ and Ar²⁶ one of an aryl group having 6 to 25 carbon atoms and a heteroaryl group having 5 to 9 carbon atoms, and
Y is one of a bivalent aromatic hydrocarbon group having 6 to 25 carbon atoms and a bivalent heterocyclic group having 5 to 10 carbon atoms.

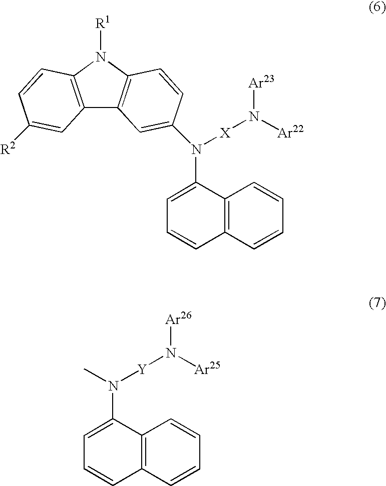

9. A material for a light emitting element represented by General Formula 8,
wherein R¹ is one selected from the group consisting of hydrogen, an alkyl group having 1 to 6 carbon atoms, an aryl group having 6 to 25 carbon atoms, a heteroaryl group having 5 to 9 carbon atoms, an arylalkyl group, and an acyl group having 1 to 7 carbon atoms, and
wherein R² is one selected from the group consisting of hydrogen, an alkyl group having 1 to 6 carbon atoms, and a substituent represented by Structural Formula 9.

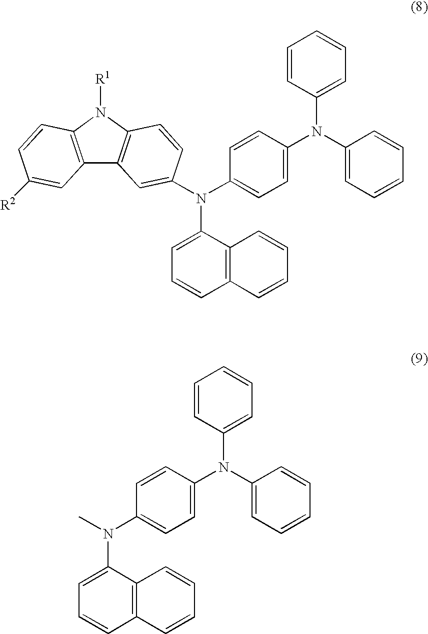

10. A light emitting element comprising:
a layer containing a light emitting substance between a pair of electrodes, wherein the layer containing the light emitting element contains the material for a light emitting element according to claim 7.

11. A light emitting element comprising:
a layer containing a light emitting substance between a pair of electrodes,
wherein the layer containing the light emitting element contains the material for a light emitting element according to claim 8.

12. A light emitting element comprising:
a layer containing a light emitting substance between a pair of electrodes,
wherein the layer containing the light emitting element contains the material for a light emitting element according to claim 9.

13. A light emitting element comprising:
a first electrode;
a second electrode;
a layer containing a light emitting substance between the first electrode and the second electrode; and
a light emitting layer included in the layer containing the light emitting substance,
wherein a layer containing the material for a light emitting element according to claim 7 is provided on a first electrode side of the light emitting layer, and
wherein the light emitting substance emits light when a voltage is applied such that a potential of the first electrode is higher than that of the second electrode.

14. A light emitting element comprising:
a first electrode;
a second electrode;
a layer containing a light emitting substance between the first electrode and the second electrode; and
a light emitting layer included in the layer containing the light emitting substance,
wherein a layer containing the material for a light emitting element according to claim 8 is provided on a first electrode side of the light emitting layer, and
wherein the light emitting substance emits light when a voltage is applied such that a potential of the first electrode is higher than that of the second electrode.

15. A light emitting element comprising:
a first electrode;
a second electrode;
a layer containing a light emitting substance between the first electrode and the second electrode; and
a light emitting layer included in the layer containing the light emitting substance,
wherein a layer containing the material for a light emitting element according to claim 9 is provided on a first electrode side of the light emitting layer, and
wherein the light emitting substance emits light when a voltage is applied such that a potential of the first electrode is higher than that of the second electrode.

16. A light emitting element comprising:
a layer containing a light emitting substance between a pair of electrodes, and
a light emitting layer included in the layer containing the light emitting substance,
wherein the light emitting layer contains the material for a light emitting element according to claim 7.

17. A light emitting element comprising:
a layer containing a light emitting substance between a pair of electrodes, and
a light emitting layer included in the layer containing the light emitting substance,
wherein the light emitting layer contains the material for a light emitting element according to claim 8.

18. A light emitting element comprising:
a layer containing a light emitting substance between a pair of electrodes, and
a light emitting layer included in the layer containing the light emitting substance,
wherein the light emitting layer contains the material for a light emitting element according to claim 9.

19. A light emitting element, comprising
a layer containing a light emitting substance between a pair of electrodes,
wherein the light emitting substance is the material for a light emitting element according to claim 7.

20. A light emitting element, comprising
a layer containing a light emitting substance between a pair of electrodes,
wherein the light emitting substance is the material for a light emitting element according to claim 8.

21. A light emitting element, comprising
a layer containing a light emitting substance between a pair of electrodes,
wherein the light emitting substance is the material for a light emitting element according to claim 9.

22. A light emitting device comprising:
the light emitting element according to claim 10, and
control means which controls light emission of the light emitting element.

23. A light emitting device comprising:
the light emitting element according to claim 11, and
control means which controls light emission of the light emitting element.

24. A light emitting device comprising:
the light emitting element according to claim 12, and
control means which controls light emission of the light emitting element.

25. A light emitting device comprising:
the light emitting element according to claim 13, and
control means which controls light emission of the light emitting element.

26. A light emitting device comprising:
the light emitting element according to claim 14, and
control means which controls light emission of the light emitting element.

27. A light emitting device comprising:
the light emitting element according to claim 15, and
control means which controls light emission of the light emitting element.

28. A light emitting device comprising:
the light emitting element according to claim 16, and
control means which controls light emission of the light emitting element.

29. A light emitting device comprising:
the light emitting element according to claim 17, and
control means which controls light emission of the light emitting element.

30. A light emitting device comprising:
the light emitting element according to claim 18, and
control means which controls light emission of the light emitting element.

31. A light emitting device comprising:
the light emitting element according to claim 19, and
control means which controls light emission of the light emitting element.

32. A light emitting device comprising:
the light emitting element according to claim 20, and
control means which controls light emission of the light emitting element.

33. A light emitting device comprising:
the light emitting element according to claim 21, and
control means which controls light emission of the light emitting element.

34. An electronic device comprising a display portion,
wherein the display portion includes the light emitting element according to claim 10 and control means which controls light emission of the light emitting element.

35. An electronic device comprising a display portion,
wherein the display portion includes the light emitting element according to claim 11 and control means which controls light emission of the light emitting element.

36. An electronic device comprising a display portion,
wherein the display portion includes the light emitting element according to claim 12 and control means which controls light emission of the light emitting element.

37. An electronic device comprising a display portion,
wherein the display portion includes the light emitting element according to claim 13 and control means which controls light emission of the light emitting element.

38. An electronic device comprising a display portion,
wherein the display portion includes the light emitting element according to claim 14 and control means which controls light emission of the light emitting element.

39. An electronic device comprising a display portion,
wherein the display portion includes the light emitting element according to claim 15 and control means which controls light emission of the light emitting element.

40. An electronic device comprising a display portion,
wherein the display portion includes the light emitting element according to claim 16 and control means which controls light emission of the light emitting element.

41. An electronic device comprising a display portion,
wherein the display portion includes the light emitting element according to claim 17 and control means which controls light emission of the light emitting element.

42. An electronic device comprising a display portion,
wherein the display portion includes the light emitting element according to claim 18 and control means which controls light emission of the light emitting element.

43. An electronic device comprising a display portion,
wherein the display portion includes the light emitting element according to claim 19 and control means which controls light emission of the light emitting element.

44. An electronic device comprising a display portion,
wherein the display portion includes the light emitting element according to claim 20 and control means which controls light emission of the light emitting element.

45. An electronic device comprising a display portion,
wherein the display portion includes the light emitting element according to claim 21 and control means which controls light emission of the light emitting element.
